(19) United States
(12) Patent Application Publication
(10) Pub. No.: US 2002/0103361 A1
(43) Pub. Date: Aug. 1, 2002

(54) Mammalian chemokines; receptors; reagents; uses

(76) Inventors: Constance F. Huffine, San Francisco, CA (US); Devora L. Rossi, San Francisco, CA (US); Myriam Capone, St. Alban Leysse (FR); Joseph A. Hedrick, Plainsboro, NJ (US); Alain Vicari, Lyon (FR); Daniel M. Gorman, Newark, CA (US); Albert Zlotnik, Palo Alto, CA (US)

Correspondence Address: DNAX RESEARCH INSTITUTE, LEGAL DEPARTMENT, 901 CALIFORNIA AVENUE, PALO ALTO, CA, 94304, US

(21) Appl. No.: 09/943,718

(22) Filed: Aug. 30, 2001

Related U.S. Application Data
(63) Continuation of application No. 09/009,817, filed on Jan. 20, 1998, now abandoned.
(60) Provisional application No. 60/035,956, filed on Jan. 21, 1997.

(51) Int. Cl. C07H 21/04; C12P 21/04; C07K 14/405; C12N 5/06

(52) U.S. Cl. 536/23.5; 530/350; 435/69.7; 435/325; 435/320.1

(57) ABSTRACT

Novel chemokines and 7 transmembrane receptors from mammals, reagents related thereto, including purified proteins, specific antibodies, and nucleic acids encoding said chemokines or receptors. Methods of using said reagents and diagnostic kits are also provided.

The present filing is a conversion of provisional U.S. patent application Ser. No. 60/035,956, filed Jan. 21, 1997, which is incorporated herein by reference, to a U.S. Utility patent application.

### FIELD OF THE INVENTION

The present invention relates to compositions related to proteins which function in controlling physiology, development, and/or differentiation of mammalian cells. In particular, it provides proteins which are implicated in the regulation of physiology, development, differentiation, or function of various cell types, e.g., chemokines, 7 transmembrane receptors, reagents related to each, e.g., antibodies or nucleic acids encoding them, and uses thereof.

### BACKGROUND OF THE INVENTION

The circulating component of the mammalian circulatory system comprises various cell types, including red and white blood cells of the erythroid and myeloid cell lineages. See, e.g., Rapaport (1987) Introduction to Hematology (2d ed.) Lippincott, Philadelphia, Pa.; Jandl (1987) Blood: Textbook of Hematology, Little, Brown and Co., Boston, Mass.; and Paul (ed.) (1993) Fundamental Immunology (3d ed.) Raven Press, N.Y.

For some time, it has been known that the mammalian immune response is based on a series of complex cellular interactions, called the “immune network.” Recent research has provided new insights into the inner workings of this network. While it remains clear that much of the response does, in fact, revolve around the network-like interactions of lymphocytes, macrophages, granulocytes, and other cells, immunologists now generally hold the opinion that soluble proteins, known as lymphokines, cytokines, or monokines, play a critical role in controlling these cellular interactions. Thus, there is considerable interest in the isolation, characterization, and mechanisms of action of cell modulatory factors, an understanding of which should lead to significant advancements in the diagnosis and therapy of numerous medical abnormalities, e.g., immune system and other disorders.

Lymphokines apparently mediate cellular activities in a variety of ways. They have been shown to support the proliferation, growth, and differentiation of the pluripotential hematopoietic stem cells into vast numbers of progenitors comprising diverse cellular lineages making up a complex immune system. These interactions between the cellular components are necessary for a healthy immune response. These different cellular lineages often respond in a different manner when lymphokines are administered in conjunction with other agents.

The chemokines are a large and diverse superfamily of proteins. The superfamily is subdivided into two classical branches, based upon whether the first two cysteines in the chemokine motif are adjacent (termed the “C-C” branch), or spaced by an intervening residue (“C-X-C”). A more recently identified branch of chemokines lacks two cysteines in the corresponding motif, and is represented by the chemokines known as lymphotactins. Another recently identified branch has three intervening residues between the two cysteines, e.g., CX3C chemokines. See, e.g., Schall and Bacon (1994) Current Opinion in Immunology 6:865-873; and Bacon and Schall (1996) Int. Arch. Allergy & Immunol. 109:97-109.

The superfamily of G-protein coupled (or linked) receptors (GPCR, or GPLR) also encompasses the chemokine receptors. As a class, these receptors are integral membrane proteins characterized by amino acid sequences which contain seven hydrophobic domains. See, e.g., Ruffolo and Hollinger (eds. 1995) G-Protein Coupled Transmembrane Signaling Mechanisms CRC Press, Boca Raton, Fla.; Watson and Arkinstall (1994) The G-Protein Linked Receptor FactsBook Academic Press, San Diego, Calif.; Peroutka (ed. 1994) G Protein-Coupled Receptors CRC Press, Boca Raton, Fla.; Houslay and Milligan (1990) G-Proteins as Mediators of Cellular Signaling Processes Wiley and Sons, New York, N.Y.; and Dohlman, et al. (1991) Ann. Rev. Biochem. 60:653-688. These hydrophobic domains are predicted to represent transmembrane spanning regions of the proteins. These GPCRs are found in a wide range of organisms and are typically involved in the transmission of signals to the interior of the cell, e.g., through interaction, e.g., with heterotrimeric G-proteins. They respond to a wide and diverse range of agents including lipid analogs, amino acid derivatives, small peptides, and other molecules.

The presumed transmembrane segments are typically 20-25 amino acids in length. Based upon models and data on bacteriorhodopsin, these regions are predicted to be α-helices and be oriented to form a ligand binding pocket. See, e.g., Findley, et al. (1990) Trends Pharmacol. Sci. 11:492-499. Other data suggest that the amino termini of the proteins are extracellular, and the carboxy termini are intracellular. See, e.g., Lodish, et al. (1995) Molecular Cell Biology 3d ed., Scientific American, New York; and Watson and Arkinstall (1994) The G-Protein Linked Receptor FactsBook Academic Press, San Diego, Calif. Phosphorylation cascades have been implicated in the signal transduction pathway of these receptors.

Although the full spectrum of biological activities mediated by these 7 transmembrane receptors has not been fully determined, chemoattractant effects are recognized. Chemokine receptors are notable members of the GPCR family. See, e.g., Samson, et al. (1996) Biochemistry 35:3362-3367; and Rapport, et al. (1996) J. Leukocyte Biology 59:18-23. The best known biological functions of these chemokine molecules relate to chemoattraction of leukocytes. However, new chemokines and receptors are being discovered, and their biological effects on the various cells responsible for immunological responses are topics of continued study.

Many factors have been identified which influence the differentiation process of precursor cells, or regulate the physiology or migration properties of specific cell types. These observations indicate that other factors exist whose functions in immune function were heretofore unrecognized. These factors provide for biological activities whose spectra of effects may be distinct from known differentiation or activation factors. The absence of knowledge about the structural, biological, and physiological properties of the regulatory factors which regulate cell physiology in vivo prevents the modulation of the effects of such factors.

In addition, other factors exist whose functions in hematopoiesis, neural function, immune development, and leukocyte trafficking were heretofore unrecognized. These receptors mediate biological activities whose spectra of effects are distinct from known differentiation, activation, or other signaling factors. The absence of knowledge about the structural, biological, and physiological properties of the receptors which regulate cell physiology, development, or function prevents the modification of the effects of such factors.

Thus, medical conditions where regulation of the development or physiology of relevant cells is required remain unmanageable.

### SUMMARY OF THE INVENTION

The present invention is based, in part, upon the discovery of new genes encoding various chemokines, e.g., those designated CKDLR20.1, which encode a CXC chemokine; or 7 transmembrane receptors, e.g., those designated 69A08, which are exemplified by a mouse clone; and HSD12, which are exemplified by a human clone. Each of the 7 transmembrane receptors is probably a G-protein coupled (or linked) receptors (GPCR or GPLR), though a ligand for each has not yet been identified.

The invention also provides mutations (muteins) of the respective natural sequences, fusion proteins, chemical mimetics, antibodies, and other structural or functional analogs. It is also directed to isolated nucleic acids, e.g., genes encoding respective proteins of the invention. Various uses of these different protein, antibody, or nucleic acid compositions are also provided.

The present invention provides a composition selected from the group of: a substantially pure antigenic polypeptide comprising sequence from a CKDLR20.1; a 69A08; or an HSD12; a binding composition comprising an antigen binding portion of an antibody specific for binding to such an antigenic polypeptide; a nucleic acid encoding such an antigenic polypeptide; and a fusion protein comprising at least two non-overlapping segments of at least 10 amino acids of such an antigenic polypeptide.

In certain embodiments of the antigenic polypeptide, it is from a warm blooded animal, e.g., a mouse or human; it comprises a sequence of Tables 1-3; it exhibits a post-translational modification pattern distinct from a natural form of said polypeptide; it is detectably labeled; or it is made by expression of a recombinant nucleic acid. In other embodiments, a sterile form is provided, including, e.g., composition comprising the polypeptide and an acceptable carrier. A detection kit comprising a compartment or container holding such an antigenic polypeptide is also provided.

In other binding composition forms, e.g., antibody embodiments, the polypeptide is a mouse or human protein; the antibody is raised against a peptide sequence of Tables 1-3; the antibody is a monoclonal antibody; the binding composition is fused to a heterologous protein, or is detectably labeled. An alternative embodiment is a binding compound comprising an antigen binding fragment of the antibody described. Also provided is a detection kit comprising such a binding compound. With the antibodies are provided methods of purifying a polypeptide using the binding compound or antibody to specifically separate the polypeptides from others.

With the binding compositions are provided methods, e.g., of producing an antigen:antibody complex, comprising contacting: a CKDLR20.1 protein or peptide with a specific antibody; a 69A08 protein or peptide with a specific antibody; or an HSD12 protein or peptide with a specific antibody; thereby allowing the complex to form. Preferably, the method is one wherein: the complex is purified from other chemokine or chemokine receptor; the complex is purified from other antibody; the contacting is with a sample comprising a CKDLR20.1 chemokine antigen; the contacting is with a sample comprising either 69A08 or HSD12 receptor antigen; the contacting allows quantitative detection of the antigen; the contacting is with a sample comprising the antibody; or the contacting allows quantitative detection of the antibody.

Nucleic acid embodiments are provided, e.g., where the nucleic acid is in an expression vector and: encodes a polypeptide from a mouse or human; comprises a sequence of a mature protein of Tables 1-3; or comprises a deoxyribonucleic acid nucleotide. The invention also provides a kit with such nucleic acids.

With nucleic acids are provided fusion proteins, comprising: a sequence of Tables 1-3; and/or sequence of another chemokine or 7 transmembrane receptor, as appropriate. Also provided is a cell comprising a recombinant nucleic acid, as described, and methods of producing a polypeptide comprising expressing the nucleic acid in an expression system.

Other embodiments include methods of producing a ligand:receptor complex, comprising contacting: a protein made by expression of a CKDLR20.1 nucleic acid with a G protein coupled receptor; a protein or peptide made by expression of a 69A08 nucleic acid with a chemokine or ligand; or a protein or peptide made by expression of an HSD12 nucleic acid with a chemokine or ligand; thereby allowing the complex to form. In certain preferred embodiments of the method: the complex results in a Ca++ flux; the G protein coupled receptor is on a cell; the complex results in a physiological change in a cell expressing the receptor or protein; the 69A08 or HSD12 protein is on a cell; the contacting is with a sample comprising a chemical antagonist to block production of the complex; or the contacting allows quantitative detection of ligand.

The invention further provides methods of modulating physiology or development of a cell, with a step of contacting that cell with a composition comprising an agonist or antagonist of the receptor. Ordinarily, the cell is a neuron, macrophage, or lymphocyte. Various physiological effects to be modulated include a cellular calcium flux, a chemoattractant response, cellular morphology modification responses, phosphoinositide lipid turnover, or an antiviral response.

### DETAILED DESCRIPTION OF THE PREFERRED EMBODIMENTS

Outline

I. General

II. Purified Chemokines; Receptors

A. physical properties

B. biological properties

III. Physical Variants

A. sequence variants, fragments

B. post-translational variants

1. glycosylation

2. others

IV. Functional Variants

A. analogs; fragments

1. agonists

2. antagonists

B. mimetics

1. protein

2. chemicals

C. species variants

V. Antibodies

A. polyclonal

B. monoclonal

C. fragments, binding compositions, fusion proteins

VI. Nucleic Acids

A. natural isolates; methods

B. synthetic genes

C. methods to isolate

VII. Making Chemokines, Receptors; Mimetics

A. recombinant methods

B. synthetic methods

C. natural purification

VIII. Uses

A. diagnostic

B. therapeutic

IX. Kits

A. nucleic acid reagents

B. protein reagents

C. antibody reagents

X. Receptors; Ligands

I. General

The present invention provides DNA sequences encoding various mammalian proteins, including chemokines, or which exhibit structural properties characteristic of a 7 transmembrane receptor. See, e.g., Ruffolo and Hollinger (eds. 1995) G-Protein Coupled Transmembrane Signaling Mechanisms CRC Press, Boca Raton, Fla.; Watson and Arkinstall (1994) The G-Protein Linked Receptor FactsBook Academic Press, San Diego, Calif.; Peroutka (ed. 1994) G Protein-Coupled Receptors CRC Press, Boca Raton, Fla.; Houslay and Milligan (1990) G-Proteins as Mediators of Cellular Signaling Processes Wiley and Sons, New York, N.Y. Certain human and mouse embodiments are described herein.

Among the many types of ligands which mediate biology via these receptors are chemokines and certain proteases. Chemokines play an important role in immune and inflammatory responses by inducing migration and adhesion of leukocytes. See, e.g., Schall (1991) Cytokine 3:165-183; and Thomson (ed.) The Cytokine Handbook Academic Press, NY. Chemokines are secreted by activated leukocytes and act as a chemoattractant for a variety of cells which are involved in inflammation. Besides chemoattractant properties, chemokines have been shown to induce other biological responses, e.g., modulation of second messenger levels such as Ca⁺⁺; inositol phosphate pool changes (see, e.g., Berridge (1993) Nature 361:315-325 or Billah and Anthes (1990) Biochem. J. 269:281-291); cellular morphology modification responses; phosphoinositide lipid turnover; possible antiviral responses; and others. Thus, the chemokines provided herein may, alone or in combination with other therapeutic reagents, have advantageous combination effects.

Moreover, there are reasons to suggest that chemokines may have effects on other cell types, e.g., attraction or activation of monocytes, dendritic cells, T cells, eosinophils, and/or perhaps on basophils and/or neutrophils. They may also have chemoattractive effects on various neural cells including, e.g., dorsal root ganglia neurons in the peripheral nervous system and/or central nervous system neurons.

G-protein coupled receptors, e.g., chemokine receptors, are important in the signal transduction mechanisms mediated by their ligands. They are useful markers for distinguishing cell populations, and have been implicated as specific receptors for retroviral infections.

The chemokine superfamily was classically divided into two groups exhibiting characteristic structural motifs, the Cys-X-Cys (C-X-C) and Cys-Cys (C-C) families. These were distinguished on the basis of a single amino acid insertion between the NH-proximal pair of cysteine residues and sequence similarity. Typically, the C-X-C chemokines, i.e., IL-8 and MGSA/Gro-α act on neutrophils but not on monocytes, whereas the C-C chemokines, i.e., MIP-1α and RANTES, are potent chemoattractants for monocytes and lymphocytes but not neutrophils. See, e.g., Miller, et al. (1992) Crit. Rev. Immunol. 12:17-46. A recently isolated chemokine, lymphotactin, does not belong to either group and may constitute a first member of a third chemokine family, the C family. Lymphotactin does not have a characteristic CC or CXC motif, and acts on lymphocytes but not neutrophils and monocytes. See, e.g., Kelner et al. (1994) Science 266:1395-1399. This chemokine defines a new C-C chemokine family. Even more recently, another chemokine exhibiting a CX3C motif has been identified, which establishes a fourth structural class.

The present invention provides additional chemokine reagents, e.g., nucleic acids, proteins and peptides, antibodies, etc., related to the newly discovered chemokines designated CKDLR20.1.

In other embodiments, the invention provides two genes encoding novel G-protein coupled receptors, designated 69A08 and HSD12. Their ligands have not yet specifically been identified. However, the receptors exhibit structural features typical of known 7 transmembrane spanning receptors, which receptors include chemokine receptors. The receptors may exhibit properties of binding many different cytokines at varying specificities (shared or promiscuous binding specificity) or may exhibit high affinity for one (specific) or a subset (shared) of chemokines. Alternatively, the ligands may be other molecules, including molecules such as epinephrine, serotonin, or glucagon.

The described chemokines or receptors should be important for mediating various aspects of cellular, organ, tissue, or organismal physiology or development.

II. Purified Chemokines; Receptors

Mouse CKDLR20.1 chemokine nucleotide and amino acid sequences are shown in Table 1. The gene encodes a novel protein exhibiting structure and motifs characteristic of a chemokine. The protein exhibits an ELR motif just upstream of the CXC sequence, implicating the chemokine in pro-inflammatory immune responses. The mRNA expression appears highly restricted to lung, and is induced in infection by the parasite Nippostongylus brasiliensis.

Nucleotide and amino acid sequences of a novel GPCR, from a mouse, designated 69A08, are provided in Table 2.

Nucleotide and derived amino acid sequences of a second novel GPCR, from human, designated HSD12, are shown in Table 3. Generic descriptions of physical properties of polypeptides, nucleic acids, and antibodies, where directed to one embodiment clearly are generally applicable to other chemokines or receptors described herein.

TABLE 1
Nucleotide sequence (5′ to 3′) of a mouse embodiment of the
mammalian chemokine, designated CKDLR20.1. The corresponding amino
acid sequence is also provided. See SEQ ID NO: 1 and 2.
Complementary nucleic acid sequences may be used for many purposes,
e.g., in a PCR primer pair or as a mutagenesis primer. Fragments of
the nucleotide sequence may be used as hybridization probes, or PCR
primers, or to encode antigenic peptides. Fragments of the
polypeptide will be useful as antigenic peptides. The gene was first
found while screening a rag lung library with the human MIP-3α probe
(complete cDNA). Note ELRCLC motif at residues 2-7.

| | |
|---|---|
| ATG CTC CTG CTG GCT GTC CTT AAC CTA GGC ATC TTC GTC CGT CCC TGT | 48 |
| Met Leu Leu Leu Ala Val Leu Asn Leu Gly Ile Phe Val Arg Pro Cys | |
| -18 -15 -10 -5 | |
| GAC ACT CAA GAG CTA CGA TGT CTG TGT ATT CAG GAA CAC TCT GAA TTC | 96 |
| Asp Thr Gln Glu Leu Arg Cys Leu Cys Ile Gln Glu His Ser Glu Phe | |
| 1 5 10 | |
| ATT CCT CTC AAA CTC ATT AAA AAT ATA ATG GTG ATA TTC GAG ACC ATT | 144 |
| Ile Pro Leu Lys Leu Ile Lys Asn Ile Met Val Ile Phe Glu Thr Ile | |
| 15 20 25 30 | |
| TAC TGC AAC AGA AAG GAA GTG ATA GCA GTC CCA AAA AAT GGG AGT ATG | 192 |
| Tyr Cys Asn Arg Lys Glu Val Ile Ala Val Pro Lys Asn Gly Ser Met | |
| 35 40 45 | |
| ATT TGT TTG GAT CCT GAT GCT CCA TGG GTG AAG GCT ACT GTT GGC CCA | 240 |
| Ile Cys Leu Asp Pro Asp Ala Pro Trp Val Lys Ala Thr Val Gly Pro | |
| 50 55 60 | |
| ATT ACT AAC AGG TTC CTA CCT GAG GAC CTC AAA CAA AGG GAA TTT CCA | 288 |
| Ile Thr Asn Arg Phe Leu Pro Glu Asp Leu Lys Gln Lys Glu Phe Pro | |
| 65 70 75 | |
| CCG GCA ATG AAG CTT CTG TAT AGT GTT GAG CAT GAA AAG CCT CTA TAT | 336 |
| Pro Ala Met Lys Leu Leu Tyr Ser Val Glu His Glu Lys Pro Leu Tyr | |
| 80 85 90 | |
| CTT TCA TTT GGG AGA CCT GAG AAC AAG AGA ATA TTT CCC TTT CCA ATT | 384 |
| Leu Ser Phe Gly Arg Pro Glu Asn Lys Arg Ile Phe Pro Phe Pro Ile | |
| 95 100 105 110 | |
| CGG GAG ACC TCT AGA CAC TTT GCT GAT TTA GCT CAC AAC AGT GAT AGG | 432 |
| Arg Glu Thr Ser Arg His Phe Ala Asp Leu Ala His Asn Ser Asp Arg | |
| 115 120 125 | |
| AAT TTT CTA CGG GAC TCC AGT GAA TTC AGC TTG ACA GGC AGT GAT GCC | 480 |
| Asn Phe Leu Arg Asp Ser Ser Glu Phe Ser Leu Thr Gly Ser Asp Ala | |
| 130 135 140 | |
| TAA | 483 |

TABLE 2
Nucleotide sequence (5′ to 3′) of mammalian GPCR 69A08, from
mouse, isolated from pre-T cells. Part of this sequence has been derived
by PCR. The corresponding amino acid sequence is also provided. See SEQ
ID NO: 3 and 4. Subsequent sequencing suggests that nucleotides 158, 159,
and 276 are absent, resulting in a region of frameshift, as indicated in
SEQ ID NO: 5 and 6.

| | |
|---|---|
| ATG AAG GCC CTC TGG GTC CCA CAG TAC AAC TCA AGG AGC CGA AGT CCT | 48 |
| Met Lys Ala Leu Trp Val Pro Gln Tyr Asn Ser Arg Ser Arg Ser Pro | |
| 1 5 10 15 | |
| CAG ACA AGC CTA ATC CAC GAG GCT ACC CGG GCA AAT TCT GTG CCA ACG | 96 |
| Gln Thr Ser Leu Ile His Glu Ala Thr Arg Ala Asn Ser Val Pro Thr | |
| 20 25 30 | |
| ACA GTG ACA CGC TGG AGC TCC CGG CCA GCT CTC AAG CAC TGC TGC TGG | 144 |
| Thr Val Thr Arg Trp Ser Ser Arg Pro Ala Leu Lys His Cys Cys Trp | |
| 35 40 45 | |
| GGT GGG TCC CCA CGA AGC TGG TAC CTG CCC TCT ATG GGC TTG TGG TGG | 192 |
| Gly Gly Ser Pro Arg Ser Trp Tyr Leu Pro Ser Met Gly Leu Trp Trp | |
| 50 55 60 | |
| CTG TGG GGC TGC CTG CCA ATG GGC TGG CGC TGT GGG TGC TGG CCA CAA | 240 |
| Leu Trp Gly Cys Leu Pro Met Gly Trp Arg Cys Gly Cys Trp Pro Gln | |
| 65 70 75 80 | |
| GGG TGC CAC GCC TGC CAT CCA CCA TTC TGC TCA TGG AAC CTG GCA GTG | 288 |
| Gly Cys His Ala Cys His Pro Pro Phe Cys Ser Trp Asn Leu Ala Val | |
| 85 90 95 | |
| GCT GAT CTG CTG TTG GCC CTG GTG CTG CCA CCA CGA CTG GCT TAC CAC | 336 |
| Ala Asp Leu Leu Leu Ala Leu Val Leu Pro Pro Arg Leu Ala Tyr His | |
| 100 105 110 | |
| TTG CGT GGC CAG CGC TGG CCA TTT GGT GAG GCT GCC TGC CGG GTG GCC | 384 |
| Leu Arg Gly Gln Arg Trp Pro Phe Gly Glu Ala Ala Cys Arg Val Ala | |
| 115 120 125 | |
| ACA GCT GCC CTC TAT GGC CAC ATG TAT GGT TCA GTG TTG CTG CTG GCT | 432 |
| Thr Ala Ala Leu Tyr Gly His Met Tyr Gly Ser Val Leu Leu Leu Ala | |
| 130 135 140 | |
| GCA GTC AGC TTG GAC AGA TAC CTG GCC CTG GTG CAT CCT TTG CGG GCC | 480 |
| Ala Val Ser Leu Asp Arg Tyr Leu Ala Leu Val His Pro Leu Arg Ala | |
| 145 150 155 160 | |
| CGT GCA CTG CGT GGT CAA CGC CTC ACT ACT GGA CTC TGT TTG GTG GCC | 528 |
| Arg Ala Leu Arg Gly Gln Arg Leu Thr Thr Gly Leu Cys Leu Val Ala | |
| 165 170 175 | |
| TGG CTC TCT GCA GCC ACC CTG GCC TTG CCT CTC ACT CTG CAT CGG CAG | 576 |
| Trp Leu Ser Ala Ala Thr Leu Ala Leu Pro Leu Thr Leu His Arg Gln | |
| 180 185 190 | |
| AAC TTC CGA TTA CTG GCT CCG ATC GCA TGC TGT GTC ATG ATG CGC TGC | 624 |
| Asn Phe Arg Leu Leu Ala Pro Ile Ala Cys Cys Val Met Met Arg Cys | |
| 195 200 205 | |
| CCC TGG CTG AGC AGA ACT CCC ACT GGA GAA CGG CCT TCA TCT GCC TGG | 672 |
| Pro Trp Leu Ser Arg Thr Pro Thr Gly Glu Arg Pro Ser Ser Ala Trp | |
| 210 215 220 | |
| CTG TCC TGG GCT GCT TCC TTG CCA CTG CTG GCC ATG GGC CTG TGC TAT | 720 |
| Leu Ser Trp Ala Ala Ser Leu Pro Leu Leu Ala Met Gly Leu Cys Tyr | |
| 225 230 235 240 | |
| GGA ACC ACC CTT CGT GCA TTG GCG GCC AAT GGC CAG CGC TAC AGC CAT | 768 |
| Gly Thr Thr Leu Arg Ala Leu Ala Ala Asn Gly Gln Arg Tyr Ser His | |
| 245 250 255 | |
| GCA CTC AGA CTG ACA GCC CTG GTA CTG TTC TCG GCA GTG GCT TCT TTC | 816 |
| Ala Leu Arg Leu Thr Ala Leu Val Leu Phe Ser Ala Val Ala Ser Phe | |
| 260 265 270 | |
| ACA CCT AGC AAT GTG CTG CTG GTG CTG CAC TAT TCA AAC CCG AGC CCT | 864 |
| Thr Pro Ser Asn Val Leu Leu Val Leu His Tyr Ser Asn Pro Ser Pro | |
| 275 280 285 | |
| GAG GCC TGG GGC AAT CTC TAT GGA GCC TAT GTG CCC AGC CTG GCA CTC | 912 |
| Glu Ala Trp Gly Asn Leu Tyr Gly Ala Tyr Val Pro Ser Leu Ala Leu | |
| 290 295 300 | |
| AGC ACC CTC AAC AGC TGC GTA GAC CCT TTC ATC TAC TAC TAT GTG TCC | 960 |
| Ser Thr Leu Asn Ser Cys Val Asp Pro Phe Ile Tyr Tyr Tyr Val Ser | |
| 305 310 315 320 | |
| CAT GAG TTC AGG GAG AAG GTA CGC GCT ATG TTG TGT CGC CAG CCG GAG | 1008 |
| His Glu Phe Arg Glu Lys Val Arg Ala Met Leu Cys Arg Gln Pro Glu | |
| 325 330 335 | |
| GCC AGC AGC TCC TCT CAG GCC TCC AGG GAG GCT GGA AGC CGA GGG ACT | 1056 |
| Ala Ser Ser Ser Ser Gln Ala Ser Arg Glu Ala Gly Ser Arg Gly Thr | |
| 340 345 350 | |
| GCC ATT TGC TCC TCT ACA CTT CTG TGACTCAGCA TCAGCCTGGC AGAGGGCATC | 1110 |
| Ala Ile Cys Ser Ser Thr Leu Leu | |
| 355 360 | |
| CAGACCCCCA GCATCTACGA TGATGTAAAG AGTACCAGGG GAAGCCATGA AGGCCCTCTG | 1170 |
| GGTCCCACAG TACAACTCAA GGAGCCGAAG TCCTCAGACA AGCCTAATCC ACGAGGCTAC | 1230 |
| CCGGGCAAAT TCTGTGCCAA CGACAGTGAC ACGCTGGAGC TCCCGGCCAG CTCTCAAGCA | 1290 |
| CTGCTGCTGG GGTGGGTCCC CACGAAGCTG GTACCTGCCC TCTATGGGCT TGTGGTGGCT | 1350 |
| GTGGGGCTGC CTGCCAATGG GCTGGCGCTG TGGGTGCTGG CCACAAGGGT GCCACGCCTG | 1410 |
| CCATCCACCA TTCTGCTCAT GGAACCTGGC AGTGGCTGAT CTGCTGTTGG CCCTGGTGCT | 1470 |
| GCCACCACGA CTGGCTTACC ACTTGCGTGG CCAGCGCTGG CCATTTGGTG AGGCTGCCTG | 1530 |
| CCGGGTGGCC ACAGCTGCCC TCTATGGCCA CATGTATGGT TCAGTGTTGC TGCTGGCTGC | 1590 |
| AGTCAGCTTG GACAGATACC TGGCCCTGGT GCATCCTTTG CGGGCCCGTG CACTGCGTGG | 1650 |
| TCAACGCCTC ACTACTGGAC TCTGTTTGGT GGCCTGGCTC TCTGCAGCCA CCCTGGCCTT | 1710 |
| GCCTCTCACT CTGCATCGGC AGAACTTCCG ATTACTGGCT CCGATCGCAT GCTGTGTCAT | 1770 |
| GATGCGCTGC CCCTGGCTGA GCAGAACTCC CACTGGAGAA CGGCCTTCAT CTGCCTGGCT | 1830 |
| GTCCTGGGCT GCTTCCTTGC CACTGCTGGC CATGGGCCTG TGCTATGGAA CCACCCTTCG | 1890 |
| TGCATTGGCG GCCAATGGCC AGCGCTACAG CCATGCACTC AGACTGACAG CCCTGGTACT | 1950 |
| GTTCTCGGCA GTGGCTTCTT TCACACCTAG CAATGTGCTG CTGGTGCTGC ACTATTCAAA | 2010 |
| CCCGAGCCCT GAGGCCTGGG GCAATCTCTA TGGAGCCTAT GTGCCCAGCC TGGCACTCAG | 2070 |
| CACCCTCAAC AGCTGCGTAG ACCCTTTCAT CTACTACTAT GTGTCCCATG AGTTCAGGGA | 2130 |
| GAAGGTACGC GCTATGTTGT GTCGCCAGCC GGAGGCCAGC AGCTCCTCTC AGGCCTCCAG | 2190 |
| GGAGGCTGGA AGCCGAGGGA CTGCCATTTG CTCCTCTACA CTTCTGTGAC TGGTAGCTGA | 2250 |
| GGTGGGAAGG GGGCATTCTG GCTTGACTGG GTCTCCCCTT AAACTACATC CCTCTTGAAC | 2310 |
| CCTCAGGACA TGACCTTATT TGGATATGCA GTTGGTGCGA CCTTCATTAG TGGAGCTGAG | 2370 |
| GTCCACTGGA AATGCTTTTG TAAAAGGTCT GGGTACTATA CGTCTGTCAC TCCAGCACTA | 2430 |
| GGGAGGTGGA GAAGAGGATC AGGAGTTCAG GATTATCTTT GACTGTAGTG AATTTGGAGC | 2490 |
| TAGGCTGGGC TATGTGAGAG TCCAGAGGCA GAAAGGAGTT ATGAGGTCAC TAGCTAGAGG | 2550 |
| ATGCTGAGAA ACCAGAATGG ATTTCCCCTT AGAGCTTC | 2588 |
| Revised sequence in coding region (SEQ ID NO: 5 and 6): | |
| ATG AAG GCC CTC TGG GTC CCA CAG TAC AAC TCA AGG AGC CGA AGT CCT | 48 |
| Met Lys Ala Leu Trp Val Pro Gln Tyr Asn Ser Arg Ser Arg Ser Pro | |
| 1 5 10 15 | |
| CAG ACA AGC CTA ATC CAC GAG GCT ACC CGG GCA AAT TCT GTG CCA ACG | 96 |
| Gln Thr Ser Leu Ile His Glu Ala Thr Arg Ala Asn Ser Val Pro Thr | |
| 20 25 30 | |
| ACA GTG ACA CGC TGG AGC TCC CGG CCA GCT CTC AAG CAC TGC TGC TGG | 144 |
| Thr Val Thr Arg Trp Ser Ser Arg Pro Ala Leu Lys His Cys Cys Trp | |
| 35 40 45 | |
| GGT GGG TCC CCA CAG CTG GTA CCT GCC CTC TAT GGG CTT GTG GTG GCT | 192 |
| Gly Gly Ser Pro Gln Leu Val Pro Ala Leu Tyr Gly Leu Val Val Ala | |
| 50 55 60 | |
| GTG GGG CTG CCT GCC AAT GGG CTG GCG CTG TGG GTG CTG GCC ACA AGG | 240 |
| Val Gly Leu Pro Ala Asn Gly Leu Ala Leu Trp Val Leu Ala Thr Arg | |
| 65 70 75 80 | |
| GTG CCA CGC CTG CCA TCC ACC ATT CTG CTC ATG AAC CTG GCA GTG GCT | 288 |
| Val Pro Arg Leu Pro Ser Thr Ile Leu Leu Met Asn Leu Ala Val Ala | |
| 85 90 95 | |
| GAT CTG CTG TTG GCC CTG GTG CTG CCA CCA CGA CTG GCT TAC CAC TTG | 336 |
| Asp Leu Leu Leu Ala Leu Val Leu Pro Pro Arg Leu Ala Tyr His Leu | |
| 100 105 110 | |
| CGT GGC CAG CGC TGG CCA TTT GGT GAG GCT GCC TGC CGG GTG GCC ACA | 384 |
| Arg Gly Gln Arg Trp Pro Phe Gly Glu Ala Ala Cys Arg Val Ala Thr | |
| 115 120 125 | |
| GCT GCC CTC TAT GGC CAC ATG TAT GGT TCA GTG TTG CTG CTG GCT GCA | 432 |
| Ala Ala Leu Tyr Gly His Met Tyr Gly Ser Val Leu Leu Leu Ala Ala | |
| 130 135 140 | |
| GTC AGC TTG GAC AGA TAC CTG GCC CTG GTG CAT CCT TTG CGG GCC CGT | 480 |
| Val Ser Leu Asp Arg Tyr Leu Ala Leu Val His Pro Leu Arg Ala Arg | |
| 145 150 155 160 | |
| GCA CTG CGT GGT CAA CGC CTC ACT ACT GGA CTC TGT TTG GTG GCC TGG | 528 |
| Ala Leu Arg Gly Gln Arg Leu Thr Thr Gly Leu Cys Leu Val Ala Trp | |
| 165 170 175 | |
| CTC TCT GCA GCC ACC CTG GCC TTG CCT CTC ACT CTG CAT CGG CAG AAC | 576 |
| Leu Ser Ala Ala Thr Leu Ala Leu Pro Leu Thr Leu His Arg Gln Asn | |
| 180 185 190 | |
| TTC CGA TTA CTG GCT CCG ATC GCA TGC TGT GTC ATG ATG CGC TGC CCC | 624 |
| Phe Arg Leu Leu Ala Pro Ile Ala Cys Cys Val Met Met Arg Cys Pro | |
| 195 200 205 | |
| TGG CTG AGC AGA ACT CCC ACT GGA GAA CGG CCT TCA TCT GCC TGG CTG | 672 |
| Trp Leu Ser Arg Thr Pro Thr Gly Glu Arg Pro Ser Ser Ala Trp Leu | |
| 210 215 220 | |
| TCC TGG GCT GCT TCC TTG CCA CTG CTG GCC ATG GGC CTG TGC TAT GGA | 720 |
| Ser Trp Ala Ala Ser Leu Pro Leu Leu Ala Met Gly Leu Cys Tyr Gly | |
| 225 230 235 240 | |
| ACC ACC CTT CGT GCA TTG GCG GCC AAT GGC CAG CGC TAC AGC CAT GCA | 768 |
| Thr Thr Leu Arg Ala Leu Ala Ala Asn Gly Gln Arg Tyr Ser His Ala | |
| 245 250 255 | |
| CTC AGA CTG ACA GCC CTG GTA CTG TTC TCG GCA GTG GCT TCT TTC ACA | 816 |
| Leu Arg Leu Thr Ala Leu Val Leu Phe Ser Ala Val Ala Ser Phe Thr | |
| 260 265 270 | |
| CCT AGC AAT GTG CTG CTG GTG CTG CAC TAT TCA AAC CCG AGC CCT GAG | 864 |
| Pro Ser Asn Val Leu Leu Val Leu His Tyr Ser Asn Pro Ser Pro Glu | |
| 275 280 285 | |
| GCC TGG GGC AAT CTC TAT GGA GCC TAT GTG CCC AGC CTG GCA CTC AGC | 912 |
| Ala Trp Gly Asn Leu Tyr Gly Ala Tyr Val Pro Ser Leu Ala Leu Ser | |
| 290 295 300 | |
| ACC CTC AAC AGC TGC GTA GAC CCT TTC ATC TAC TAC TAT GTG TCC CAT | 960 |
| Thr Leu Asn Ser Cys Val Asp Pro Phe Ile Tyr Tyr Tyr Val Ser His | |
| 305 310 315 320 | |
| GAG TTC AGG GAG AAG GTA CGC GCT ATG TTG TGT CGC CAG CCG GAG GCC | 1008 |
| Glu Phe Arg Glu Lys Val Arg Ala Met Leu Cys Arg Gln Pro Glu Ala | |
| 325 330 335 | |
| AGC AGC TCC TCT CAG GCC TCC AGG GAG GCT GGA AGC CGA GGG ACT GCC | 1056 |
| Ser Ser Ser Ser Gln Ala Ser Arg Glu Ala Gly Ser Arg Gly Thr Ala | |
| 340 345 350 | |
| ATT TGC TCC TCT ACA CTT CTG TGA | 1080 |
| Ile Cys Ser Ser Thr Leu Leu | |
| 355 | |

TABLE 3

Nucleotide sequence (5′ to 3′) of mammalian GPCR, HSD12, from
human. See SEQ ID NO: 7 and 8.

| | |
|---|---|
| GCAGGAGGGG GTGCGAGGCT AGCCACGCAG GCGGGGCCCT GGGTCATTTT AAACTCTCAG | 60 |
| AGTGAACGTC TTGATAGGAC CGACAAGACG CATGACATGT ACTTAGAAAG CTTATCTTAG | 120 |
| AGCCACACTG AGATTGGAAC CCGCAAAAT ATG CCA GGA AAC GCC ACC CCA GTG | 173 |
| Met Pro Gly Asn Ala Thr Pro Val | |
| 1 5 | |
| ACC ACC ACT GCC CCG TGG GCC TCC CTG GGC CTC TCC GCC AAG ACC TGC | 221 |
| Thr Thr Thr Ala Pro Trp Ala Ser Leu Gly Leu Ser Ala Lys Thr Cys | |
| 10 15 20 | |
| AAC AAC GTG TCC TTC GAA GAG AGC AGG ATA GTC CTG GTC GTG GTG TAC | 269 |
| Asn Asn Val Ser Phe Glu Glu Ser Arg Ile Val Leu Val Val Val Tyr | |
| 25 30 35 40 | |
| AGC GCG GTG TGC ACG CTG GGG GTG CCG GCC AAC TGC CTG ACT GCG TGG | 317 |
| Ser Ala Val Cys Thr Leu Gly Val Pro Ala Asn Cys Leu Thr Ala Trp | |
| 45 50 55 | |
| CTG GCG CTG CTG CAG GTA CTG CAG GGC AAC GTG CTG GCC GTC TAC CTG | 365 |
| Leu Ala Leu Leu Gln Val Leu Gln Gly Asn Val Leu Ala Val Tyr Leu | |
| 60 65 70 | |
| CTC TGC CTG GCA CTC TGC GAG CTG CTG TAC ACA GGC ACG CTG CCA CTC | 413 |
| Leu Cys Leu Ala Leu Cys Glu Leu Leu Tyr Thr Gly Thr Leu Pro Leu | |
| 75 80 85 | |
| TGG GTC ATC TAT ATC CGC AAC CAG CAC CGC TGG ACC CTA GGC CTG CTG | 461 |
| Trp Val Ile Tyr Ile Arg Asn Gln His Arg Trp Thr Leu Gly Leu Leu | |
| 90 95 100 | |
| GCC TGC AAG GTG ACC GCC TAC ATC TTC TTC TGC AAC ATC TAC GTC AGC | 509 |
| Ala Cys Lys Val Thr Ala Tyr Ile Phe Phe Cys Asn Ile Tyr Val Ser | |
| 105 110 115 120 | |
| ATC CTC TTC CTG TGC TGC ATC TCC TGC GAC CGC TTC GTG GCC GTG GTG | 557 |
| Ile Leu Phe Leu Cys Cys Ile Ser Cys Asp Arg Phe Val Ala Val Val | |
| 125 130 135 | |
| TAC GCG CTG GAG AGT CGG GGC CGC CGC CGC CGG AGG ACC GCC ATC CTC | 605 |
| Tyr Ala Leu Glu Ser Arg Gly Arg Arg Arg Arg Arg Thr Ala Ile Leu | |
| 140 145 150 | |
| ATC TCC GCC TGC ATC TTC ATC CTC GTC GGG ATC GTT CAC TAC CCG GTG | 653 |
| Ile Ser Ala Cys Ile Phe Ile Leu Val Gly Ile Val His Tyr Pro Val | |
| 155 160 165 | |
| TTC CAG ACG GAA GAC AAG GAG ACC TGC TTT GAC ATG CTG CAG ATG GAC | 701 |
| Phe Gln Thr Glu Asp Lys Glu Thr Cys Phe Asp Met Leu Gln Met Asp | |
| 170 175 180 | |
| AGC AGG ATT GCC GGG TAC TAC TAC GCC AGG TTC ACC GTT GGC TTT GCC | 749 |
| Ser Arg Ile Ala Gly Tyr Tyr Tyr Ala Arg Phe Thr Val Gly Phe Ala | |
| 185 190 195 200 | |
| ATC CCT CTC TCC ATC ATC GCC TTC ACC AAC CAC CGG ATT TTC AGG AGC | 797 |
| Ile Pro Leu Ser Ile Ile Ala Phe Thr Asn His Arg Ile Phe Arg Ser | |
| 205 210 215 | |
| ATC AAG CAG AGC ATG GGC TTA AGC GCT GCC CAG AAG GCC AAG GTG AAG | 845 |
| Ile Lys Gln Ser Met Gly Leu Ser Ala Ala Gln Lys Ala Lys Val Lys | |
| 220 225 230 | |
| CAC TCG GCC ATC GCG GTG GTT GTC ATC TTC CTA GTC TGC TTC GCC CCG | 893 |
| His Ser Ala Ile Ala Val Val Val Ile Phe Leu Val Cys Phe Ala Pro | |
| 235 240 245 | |
| TAC CAC CTG GTT CTC CTC GTC AAA GCC GCT GCC TTT TCC TAC TAC AGA | 941 |
| Tyr His Leu Val Leu Leu Val Lys Ala Ala Ala Phe Ser Tyr Tyr Arg | |
| 250 255 260 | |
| GGA GAC AGG AAC GCC ATG TGC GGC TTG GAG GAA AGG CTG TAC ACA GCC | 989 |
| Gly Asp Arg Asn Ala Met Cys Gly Leu Glu Glu Arg Leu Tyr Thr Ala | |
| 265 270 275 280 | |
| TCT GTG GTG TTT CTG TGC CTG TCC ACG GTG AAC GGC GTG GCT GAC CCC | 1037 |
| Ser Val Val Phe Leu Cys Leu Ser Thr Val Asn Gly Val Ala Asp Pro | |
| 285 290 295 | |
| ATT ATC TAC GTG CTG GCC ACG GAC CAT TCC CGC CAA GAA GTG TCC AGA | 1085 |
| Ile Ile Tyr Val Leu Ala Thr Asp His Ser Arg Gln Glu Val Ser Arg | |
| 300 305 310 | |
| ATC CAT AAG GGG TGG AAA GAG TGG TCC ATG AAG ACA GAC GTC ACC AGG | 1133 |
| Ile His Lys Gly Trp Lys Glu Trp Ser Met Lys Thr Asp Val Thr Arg | |
| 315 320 325 | |
| CTC ACC CAC AGC AGG GAC ACC GAG GAG CTG CAG TCG CCC GTG GCC CTT | 1181 |
| Leu Thr His Ser Arg Asp Thr Glu Glu Leu Gln Ser Pro Val Ala Leu | |
| 330 335 340 | |
| GCA GAC CAC TAC ACC TTC TCC AGG CCC GTG CAC CCA CCA GGG TCA CCA | 1229 |
| Ala Asp His Tyr Thr Phe Ser Arg Pro Val His Pro Pro Gly Ser Pro | |
| 345 350 355 360 | |
| TGC CCT GCA AAG AGG CTG ATT GAG GAG TCC TGC TGAGCCCACT GTGTGGCAGG | 1282 |
| Cys Pro Ala Lys Arg Leu Ile Glu Glu Ser Cys | |
| 365 370 | |
| GGGATGGCAG GTTGGGGGTC CTGGGGCCAG CAATGTGGTT CCTGTGCACT GAGCCCACCA | 1342 |
| GCCACAGTGC CCATGTCCCC TCTGGAAGAC AAACTACCAA TTTCTCGTTC CTGAAGCCAC | 1402 |
| TCCCTCCGTG ACCACTGGCC CCAGGCTTTC CCACATGGAA GGTGGCTGCA TGCCAAGGGG | 1462 |
| AGGAGCGACA CCTCCAGGCT TCCGGGAGCC CAGAGAGCAT GTGGCAGGCA GTGGGGCCTC | 1522 |
| TTCATCAGCA GCCTGCCTGG CTGGCTCCCT TGGCTGTGGG CAGGTAGCAC GCCTGCTGGC | 1582 |
| AGAGGTACCT GGTGGCTGCC CTGTTCGCAT CAGTGGCGAT GACTTTATTT GCGGAGCATT | 1642 |
| TCTGCAAGCG TTGCCTGGAT GCGGTGGTGC ATTGTGGGCC CTCTGGGCTC CTGCCTCAGA | 1702 |
| ATGTCAGTGA GCACCATGCT GGAGGTCACC CAGCACTGTG GCAGCGCCCA GGAGGGCATA | 1762 |
| GGGCAGCCTA CCACCTCCAA GGGGGCAGGC GCCCTCATCT GGGGTTGGGT CTGTGCTGAG | 1822 |
| CTGGAGGGCC TCTAGGGAAC CGTGGGGCAG GGTGGCCAGC TGCTGGCTCC CAGAGCGCAG | 1882 |
| CCCAGGCGTC CTCAACGGGG AGCCCCAAAT GTCCACGCCC AGAACAACAG TTGGCAGGAC | 1942 |
| AGGTGTGACA CAGCCACAGC AGAGGCAAGG GGTGCCAGGA GTCCCCAGCG GCATCCTCGG | 2002 |
| GGAGATGCTG GTGAGGGGTC CGTACAGGGT GGGGTCCCCA CCCCTAGCCC CTTACTGAGG | 2062 |
| GGGGAGTGCA GCAGTTGGCC TGCTTGTGTG GCGGAGAAAG CCAGCTCCCT GCACCCTCGG | 2122 |
| GGCTGAGTCA GATGTGGGTC TGCCGCAAAG GCCTTGCGTA GACCAGGTCA CACTGATGCC | 2182 |
| CTGGTTTCCC TATCTGTAAA ATGGGGCCAA TGACACCTAC CTCACTGGGT CACCATCGAG | 2242 |
| ATCAATCCTC CTCCCTGCCC GGACACCTCG GGCACATCGC ATGCACTCAG AGCACAGAGC | 2302 |
| CGGGCAGACG CAGCACCTGC ATGGGGAGCC CAGTGCCCGG CACAGCACAG GGGCTTCCAG | 2362 |
| GGAGGCCGCG CAGGGCCGTG GGGCTGAGCC ACGCTCTCGT TTTGTCAGGC AGCTATGCAG | 2422 |
| TTGCTCTTCC TTGTTTTTGT TTTGTTTTTG TTTTTGTTTT TAATATTTAT TTTTTTAGAG | 2482 |
| ACAGGGCCTT GCTCTGTTGC CTGGGCTGGA GAACAGTGGC ACCATCATAG CTCACTGCAG | 2542 |
| CCTCAAACTC CTGGGCTCAA GCGATCCTCC CCGCTCAGCC TCCTGAGTAG CTGGGACTAC | 2602 |
| AGGTGTGCAC CACCACACCC AGCCAAAACA GCCATCCTCC CCTTGAGAGT CATCAGAAAA | 2662 |
| ATACATTAGG AAAATGTGTT TAGAAATAAA AGCACAAGGC AGGGCAGTGC TCACGCCTGT | 2722 |
| CATCCCAGCA CTTTGGGAGG CCGAGACGGG AGGATCAGTT GAGGTCAGGA GTTTGAGACC | 2782 |
| AGCCTCGGCA ACATGGCAAA ATCTTGTCTC TTTTTTTTGG TATTAAAAAA ATCATAAAAA | 2842 |
| TAAAAGAAAT AATGCAATTT AA | 2864 |

These amino acid sequences, provided amino to carboxy, are important in providing sequence information on the chemokine ligand or receptor, allowing for distinguishing the protein from other proteins, particularly naturally occurring versions. Moreover, the sequences allow preparation of peptides to generate antibodies to recognize and distinguish such segments, and allow preparation of oligonucleotide probes, both of which are strategies for isolation, e.g., cloning, of genes encoding such sequences, or related sequences, e.g., natural polymorphic or other variants, including fusion proteins. Similarities of the chemokines have been observed with other cytokines. See, e.g., Bosenberg, et al. (1992) Cell 71:1157-1165; Huang, et. al. (1992) Molecular Biology of the Cell 3:349-362; and Pandiella, et al. (1992) J. Biol. Chem. 267:24028-24033. Likewise for the GPC receptors.

As used herein, the term “CKDLR20.1” shall encompass, when used in a protein context, a protein having mature amino acid sequence, as shown in Table 1. The invention also embraces a polypeptide comprising a significant fragment of such protein. The invention also encompasses a polypeptide which is a species counterpart, e.g., which exhibits similar sequence, and is more homologous in natural encoding sequence than other genes from that species, particularly primate species. Typically, such chemokine will also interact with its specific binding components, e.g., receptor, or antibodies which bind to it. These binding components, e.g., antibodies, typically bind to the chemokine with high affinity, e.g., at least about 100 nM, usually better than about 30 nM, preferably better than about 10 nM, and more preferably at better than about 3 nM. Homologous proteins would be found in mammalian species other than mouse, e.g., rats, dogs, cats, and primates. Non-mammalian species should also possess structurally or functionally related genes and proteins. Similar concepts apply to GPCR embodiments 69A08 and HSD12, in the context of a receptor.

The term “polypeptide” as used herein includes a significant fragment or segment, and encompasses a stretch of amino acid residues of at least about 8 amino acids, generally at least 10 amino acids, more generally at least 12 amino acids, often at least 14 amino acids, more often at least 16 amino acids, typically at least 18 amino acids, more typically at least 20 amino acids, usually at least 22 amino acids, more usually at least 24 amino acids, preferably at least 26 amino acids, more preferably at least 28 amino acids, and, in particularly preferred embodiments, at least about 30 or more amino acids, e.g., about 35, 40, 45, 50, 60, 75, 80, 100, 120, etc. Similar proteins will likely comprise a plurality of such segments. Such fragments may have ends which begin and/or end at virtually all positions, e.g., beginning at residues 1, 2, 3, etc., and ending at, e.g., 69, 68, 67, 66, etc., in all combinatorial pairs. Particularly interesting peptides have ends corresponding to structural domain boundaries, e.g., intracellular or extracellular loops of the receptor embodiments. Such peptides will typically be immunogenic peptides, or may be concatenated to generate larger polypeptides. Short peptides may be attached or coupled to a larger carrier.

The term “binding composition” refers to molecules that bind with specificity to the respective chemokine or receptor, e.g., in a ligand-receptor type fashion or an antibody-antigen interaction. These compositions may be compounds, e.g., proteins, which specifically associate with the chemokine or receptor, including natural physiologically relevant protein-protein interactions, either covalent or non-covalent. The binding composition may be a polymer, or another chemical reagent. No implication as to whether the chemokine presents a concave or convex shape in its ligand-receptor interaction is necessarily represented, other than the interaction exhibit similar specificity, e.g., specific affinity. A functional analog may be a ligand with structural modifications, or may be a wholly unrelated molecule, e.g., which has a molecular shape which interacts with the appropriate ligand binding determinants. The ligands may serve as agonists or antagonists of a physiological or natural receptor, see, e.g., Goodman, et al. (eds.) (1990) Goodman & Gilman's: The Pharmacological Bases of Therapeutics (8th ed.), Pergamon Press. The term expressly includes antibodies, polyclonal or monoclonal, which specifically bind to the respective antigen.

Substantially pure means that the protein is free from other contaminating proteins, nucleic acids, and/or other biologicals typically derived from the original source organism. Purity may be assayed by standard methods, and will ordinarily be at least about 40% pure, more ordinarily at least about 50% pure, generally at least about 60% pure, more generally at least about 70% pure, often at least about 75% pure, more often at least about 80% pure, typically at least about 85% pure, more typically at least about 90% pure, preferably at least about 95% pure, more preferably at least about 98% pure, and in most preferred embodiments, at least 99% pure. Analyses will typically be by weight, but may be by molar amounts.

Solubility of a polypeptide or fragment depends upon the environment and the polypeptide. Many parameters affect polypeptide solubility, including temperature, electrolyte environment, size and molecular characteristics of the polypeptide, and nature of the solvent. Typically, the temperature at which the polypeptide is used ranges from about 40° C. to about 65° C. Usually the temperature at use is greater than about 18° C. and more usually greater than about 22° C. For diagnostic purposes, the temperature will usually be about room temperature or warmer, but less than the denaturation temperature of components in the assay. For therapeutic purposes, the temperature will usually be body temperature, typically about 37° C. for humans, though under certain situations the temperature may be raised or lowered in situ or in vitro.

The electrolytes will usually approximate in situ physiological conditions, but may be modified to higher or lower ionic strength where advantageous. The actual ions may be modified, e.g., to conform to standard buffers used in physiological or analytical contexts.

The size and structure of the polypeptide should generally be in a substantially stable state, and usually not in a denatured state. The polypeptide may be associated with other polypeptides in a quaternary structure, e.g., to confer solubility, or associated with lipids or detergents in a manner which approximates natural lipid bilayer interactions.

The solvent will usually be a biologically compatible buffer, of a type used for preservation of biological activities, and will usually approximate a physiological solvent. Usually the solvent will have a neutral pH, typically at least about 5, preferably at least 6, and typically less than 10, preferably less than 9, and more preferably about 7.5. On some occasions, a detergent will be added, typically a mild non-denaturing one, e.g., CHS (cholesteryl hemisuccinate) or CHAPS (3-([3-cholamido-propyl]dimethylammonio)-1-propane sulfonate), or a low enough concentration as to avoid significant disruption of structural or physiological properties of the protein.

Solubility is reflected by sedimentation measured in Svedberg units, which are a measure of the sedimentation velocity of a molecule under particular conditions. The determination of the sedimentation velocity was classically performed in an analytical ultracentrifuge, but is typically now performed in a standard ultracentrifuge. See, Freifelder (1982) Physical Biochemistry (2d ed.), W. H. Freeman; and Cantor and Schimmel (1980) Biophysical Chemistry, parts 1-3, W.H. Freeman & Co., San Francisco. As a crude determination, a sample containing a putatively soluble polypeptide is spun in a standard full sized ultracentrifuge at about 50K rpm for about 10 minutes, and soluble molecules will remain in the supernatant. A soluble particle or polypeptide will typically be less than about 30S, more typically less than about 15S, usually less than about 10S, more usually less than about 6S, and, in particular embodiments, preferably less than about 4S, and more preferably less than about 3S.

III. Physical Variants

This invention also encompasses proteins or peptides having substantial amino acid sequence homology with the amino acid sequence of each respective receptor. The variants include species or polymorphic variants.

Amino acid sequence homology, or sequence identity, is determined by optimizing residue matches, if necessary, by introducing gaps as required. This changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Homologous amino acid sequences are typically intended to include natural allelic and interspecies variations in each respective protein sequence. Typical homologous proteins or peptides will have from 25-100% homology (if gaps can be introduced), to 50-100% homology (if conservative substitutions are included) with the amino acid sequence of the appropriate chemokine or receptor. Homology measures will be at least about 35%, generally at least 40%, more generally at least 45%, often at least 50%, more often at least 55%, typically at least 60%, more typically at least 65%, usually at least 70%, more usually at least 75%, preferably at least 80%, and more preferably at least 80%, and in particularly preferred embodiments, at least 85% or more. See also Needleham, et al. (1970) J. Mol. Biol. 48:443-453; Sankoff, et al. (1983) Chapter One in Time Warps, String Edits, and Macromolecules: The Theory and Practice of Sequence Comparison Addison-Wesley, Reading, Mass.; and software packages from IntelliGenetics, Mountain View, Calif.; and the University of Wisconsin Genetics Computer Group, Madison, Wis.

Each of the isolated chemokine or GPC receptor DNAs can be readily modified by nucleotide substitutions, nucleotide deletions, nucleotide insertions, and inversions of nucleotide stretches. These modifications may result in novel DNA sequences which encode these antigens, their derivatives, or proteins having similar physiological, immunogenic, or antigenic activity. These modified sequences can be used to produce mutant antigens or to enhance expression, or to introduce convenient enzyme recognition sites into the nucleotide sequence without significantly affecting the encoded protein sequence. Enhanced expression may involve gene amplification, increased transcription, increased translation, and other mechanisms. Such mutant receptor derivatives include predetermined or site-specific mutations of the respective protein or its fragments. “Mutant chemokine” encompasses a polypeptide otherwise falling within the homology definition of the chemokine as set forth above, but having an amino acid sequence which differs from that of the chemokine as found in nature, whether by way of deletion, substitution, or insertion. Likewise for the GPCRs. These include amino acid residue substitution levels from none, one, two, three, five, seven, ten, twelve, fifteen, etc. In particular, “site specific mutant” generally includes proteins having significant homology with a protein having sequences of Table 1 through 3, and as sharing various biological activities, e.g., antigenic or immunogenic, with those sequences, and in preferred embodiments contain most of the disclosed sequences, particularly those found in various warm blooded animals, e.g., mammals and birds. As stated before, it is emphasized that descriptions are generally meant to encompass the various chemokine or receptor proteins, not limited to the mouse or human embodiments specifically discussed.

Although site specific mutation sites are often predetermined, mutants need not be site specific. Chemokine or receptor mutagenesis can be conducted by making amino acid insertions or deletions. Substitutions, deletions, insertions, or combinations may be generated to arrive at a final construct. Insertions include amino- or carboxy-terminal fusions. Random mutagenesis can be conducted at a target codon and the expressed mutants can then be screened for the desired activity. Methods for making substitution mutations at predetermined sites in DNA having a known sequence are well known in the art, e.g., by M13 primer mutagenesis or polymerase chain reaction (PCR) techniques. See also Sambrook, et al. (1989) and Ausubel, et al. (1987 and Supplements). Many structural features are known about the chemokines and GPCRs which allow determination of whether specific residues are embedded into the core of the secondary or tertiary structures, or whether the residues will have relatively little effect on protein folding. Preferred positions for mutagenesis are those which do not prevent functional folding of the resulting protein.

The mutations in the DNA normally should not place coding sequences out of reading frames and preferably will not create complementary regions that could hybridize to produce secondary mRNA structure such as loops or hairpins. But certain situations exist where such problems are compensated. See, e.g., Gesteland and Atkins (1996) Ann. Rev. Biochem. 65:741-768.

The present invention also provides recombinant proteins, e.g., heterologous fusion proteins using segments from these proteins, or antibodies. A heterologous fusion protein is a fusion of proteins or segments which are naturally not normally fused in the same manner. Thus, the fusion product of an immunoglobulin with a receptor polypeptide is a continuous protein molecule having sequences fused in a typical peptide linkage, typically made as a single translation product and exhibiting properties derived from each source peptide. A similar chimeric concept applies to heterologous nucleic acid sequences.

In addition, new constructs may be made from combining similar functional or structural domains from other proteins. For example, ligand-binding or other segments may be “swapped” between different new fusion polypeptides or fragments. See, e.g., Cunningham, et al. (1989) Science 243:1330-1336; and O'Dowd, et al. (1988) J. Biol. Chem. 263:15985-15992. Thus, new chimeric polypeptides exhibiting new combinations of specificities will result from the functional linkage of ligand-binding specificities and other functional domains. Such may be chimeric molecules with mixing or matching of the various structural segments, e.g., the β-sheet or α-helix structural domains for the chemokine, or receptor segments corresponding to each of the transmembrane segments (TM1-TM7), or the intracellular (cytosolic, C1-C4) or extracellular (E1-E4) loops from the various receptor types. The C3 loop is particularly important.

The phosphoramidite method described by Beaucage and Carruthers (1981) Tetra. Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence, e.g., PCR techniques.

IV. Functional Variants

The blocking of physiological response to various embodiments of these chemokines or GPCRs may result from the inhibition of binding of the ligand to its receptor, likely through competitive inhibition. Thus, in vitro assays of the present invention will often use isolated protein, membranes from cells expressing a recombinant membrane associated receptor, e.g., ligand binding segments, or fragments attached to solid phase substrates. These assays will also allow for the diagnostic determination of the effects of either binding segment mutations and modifications, or ligand mutations and modifications, e.g., ligand analogs.

This invention also contemplates the use of competitive drug screening assays, e.g., where neutralizing binding compositions, e.g., antibodies, to antigen or receptor fragments compete with a test compound for binding to the protein. In this manner, the antibodies can be used to detect the presence of polypeptides which share one or more antigenic binding sites of the ligand and can also be used to occupy binding sites on the protein that might otherwise interact with a receptor.

Additionally, neutralizing antibodies against a specific chemokine embodiment and soluble fragments of the chemokine which contain a high affinity receptor binding site, can be used to inhibit chemokine activity in tissues, e.g., tissues experiencing abnormal physiology.

“Derivatives” of chemokine or receptor antigens include amino acid sequence mutants, glycosylation variants, and covalent or aggregate conjugates with other chemical moieties. Covalent derivatives can be prepared by linkage of functionalities to groups which are found in chemokine amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins may be important when immunogenic moieties are haptens.

In particular, glycosylation alterations are included, e.g., made by modifying the glycosylation patterns of a polypeptide during its synthesis and processing, or in further processing steps. Particularly preferred means for accomplishing this are by exposing the polypeptide to glycosylating enzymes derived from cells which normally provide such processing, e.g., mammalian glycosylation enzymes. Deglycosylation enzymes are also contemplated. Also embraced are versions of the same primary amino acid sequence which have other minor modifications, including phosphorylated amino acid residues, e.g., phosphotyrosine, phosphoserine, or phosphothreonine, or nucleoside or nucleotide derivatives, e.g., guanyl derivatized.

A major group of derivatives are covalent conjugates of the respective chemokine or receptor or fragments thereof with other proteins or polypeptides. These derivatives can be synthesized in recombinant culture such as N- or C-terminal fusions or by the use of agents known in the art for their usefulness in cross-linking proteins through reactive side groups. Preferred chemokine derivatization sites with cross-linking agents are at free amino groups, carbohydrate moieties, and cysteine residues.

Fusion polypeptides between these chemokines or receptors and other homologous or heterologous proteins, e.g., other chemokines or receptors, are also provided. Many growth factors and cytokines are homodimeric entities, and a repeat construct may have various advantages, including lessened susceptibility to proteolytic cleavage. Moreover, many cytokine receptors require dimerization to transduce a signal, and various dimeric ligands or domain repeats can be desirable. Homologous polypeptides may be fusions between different surface markers, resulting in, e.g., a hybrid protein exhibiting receptor binding specificity. Likewise, heterologous fusions may be constructed which would exhibit a combination of properties or activities of the derivative proteins. Typical examples are fusions of a reporter polypeptide, e.g., luciferase, with a segment or domain of a ligand, e.g., a receptor-binding segment, so that the presence or location of the fused ligand, or a binding composition, may be easily determined. See, e.g., Dull, et al., U.S. Pat. No. 4,859,609. Other gene fusion partners include bacterial 9-galactosidase, trpE, Protein A, β-lactamase, alpha amylase, alcohol dehydrogenase, a FLAG fusion, and yeast alpha mating factor. See, e.g., Godowski, et al. (1988) Science 241:812-816.

The phosphoramidite method described by Beaucage and Carruthers (1981) Tetra. Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence.

Such polypeptides may also have amino acid residues which have been chemically modified by phosphorylation, guanylation, sulfonation, biotinylation, or the addition or removal of other moieties, particularly those which have molecular shapes similar to phosphate or guanyl groups. In some embodiments, the modifications will be useful labeling reagents, or serve as purification targets, e.g., affinity tags as FLAG.

Fusion proteins will typically be made by either recombinant nucleic acid methods or by synthetic polypeptide methods. Techniques for nucleic acid manipulation and expression are described generally, for example, in Sambrook, et al. (1989) Molecular Cloning: A Laboratory Manual (2d ed.), Vols. 1-3, Cold Spring Harbor Laboratory. Techniques for synthesis of polypeptides are described, for example, in Merrifield (1963) J. Amer. Chem. Soc. 85:2149-2156; Merrifield (1986) Science 232: 341-347; and Atherton, et al. (1989) Solid Phase Peptide Synthesis: A Practical Approach, IRL Press, Oxford; and chemical ligation, e.g., Dawson, et al. (1994) Science 266:776-779, a method of linking long synthetic peptides by a peptide bond.

This invention also contemplates the use of derivatives of these chemokines or receptors other than variations in amino acid sequence or glycosylation. Such derivatives may involve covalent or aggregative association with chemical moieties. These derivatives generally include: (1) salts, (2) side chain and terminal residue covalent modifications, and (3) adsorption complexes, for example with cell membranes. Such covalent or aggregative derivatives are useful as immunogens, as reagents in immunoassays, or in purification methods such as for affinity purification of ligands or other binding ligands. For example, a chemokine antigen can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of anti-chemokine antibodies or its receptor. These chemokines can also be labeled with a detectable group, for example radioiodinated by the chloramine T procedure, covalently bound to rare earth chelates, or conjugated to a fluorescent moiety for use in diagnostic assays. Purification of chemokine, receptor, or binding compositions may be effected by immobilized antibodies or receptor.

Other modifications may be introduced with the goal of modifying the therapeutic pharmacokinetics or pharmacodynamics of a target chemokine. For example, certain means to minimize the size of the entity may improve its pharmacoaccessibility; other means to maximize size may affect pharmacodynamics. Similarly, changes in ligand binding kinetics or equilibrium of a receptor may be engineered.

A solubilized chemokine or receptor or appropriate fragment of this invention can be used as an immunogen for the production of antisera or antibodies specific for the ligand, receptor, or fragments thereof. The purified proteins can be used to screen monoclonal antibodies or chemokine-binding fragments prepared by immunization with various forms of impure preparations containing the protein. In particular, antibody equivalents include antigen binding fragments of natural antibodies, e.g., Fv, Fab, or F(ab)₂. Purified chemokines can also be used as a reagent to detect antibodies generated in response to the presence of elevated levels of the protein or cell fragments containing the protein, both of which may be diagnostic of an abnormal or specific physiological or disease condition. Additionally, chemokine protein fragments, or their concatenates, may also serve as immunogens to produce binding compositions, e.g., antibodies of the present invention, as described immediately below. For example, this invention contemplates antibodies raised against amino acid sequences shown in Tables 1 through 3, or proteins containing them. In particular, this invention contemplates antibodies having binding affinity to or being raised against specific fragments, e.g., those which are predicted to lie on the outside surfaces of protein tertiary structure. Similar concepts apply to antibodies specific for receptors of the invention.

The present invention contemplates the isolation of additional closely related species variants. Southern and Northern blot analysis should establish that similar genetic entities exist in other mammals, and establish the stringency of hybridization conditions to isolate such. It is likely that these chemokines and receptors are widespread in species variants, e.g., rodents, lagomorphs, carnivores, artiodactyla, perissodactyla, and primates.

The invention also provides means to isolate a group of related chemokines or receptors displaying both distinctness and similarities in structure, expression, and function. Elucidation of many of the physiological effects of the proteins will be greatly accelerated by the isolation and characterization of distinct species variants of the ligands. Related genes found, e.g., in various computer databases will also be useful, in many instances, for similar purposes with structurally related proteins. In particular, the present invention provides useful probes or search features for identifying additional homologous genetic entities in different species.

The isolated genes will allow transformation of cells lacking expression of a corresponding chemokine or receptor, e.g., either species types or cells which lack corresponding antigens and exhibit negative background activity. Expression of transformed genes will allow isolation of antigenically pure cell lines, with defined or single specie variants. This approach will allow for more sensitive detection and discrimination of the physiological effects of chemokine or receptor proteins. Subcellular fragments, e.g., cytoplasts or membrane fragments, can be isolated and used.

Dissection of critical structural elements which effect the various differentiation functions provided by ligands is possible using standard techniques of modern molecular biology, particularly in comparing members of the related class. See, e.g., the homolog-scanning mutagenesis technique described in Cunningham, et al. (1989) Science 243:1339-1336; and approaches used in O'Dowd, et al. (1988) J. Biol. Chem. 263:15985-15992; and Lechleiter, et al. (1990) EMBO J. 9:4381-4390.

In addition, various segments can be substituted between species variants to determine what structural features are important in both receptor binding affinity and specificity, as well as signal transduction. An array of different chemokine or receptor variants will be used to screen for variants exhibiting combined properties of interaction with different species variants.

Intracellular functions would probably involve segments of the receptor which are normally accessible to the cytosol. However, ligand internalization may occur under certain circumstances, and interaction between intracellular components and “extracellular” segments may occur. The specific segments of interaction of a particular chemokine with other intracellular components may be identified by mutagenesis or direct biochemical means, e.g., cross-linking or affinity methods. Structural analysis by crystallographic or other physical methods will also be applicable. Further investigation of the mechanism of signal transduction will include study of associated components which may be isolatable by affinity methods or by genetic means, e.g., complementation analysis of mutants.

Further study of the expression and control of the various chemokines or receptors will be pursued. The controlling elements associated with the proteins may exhibit differential developmental, tissue specific, or other expression patterns. Upstream or downstream genetic regions, e.g., control elements, are of interest. Differential splicing of message may lead to membrane bound forms, soluble forms, and modified versions of ligand.

Structural studies of the proteins will lead to design of new ligands or receptors, particularly analogs exhibiting agonist or antagonist properties on the receptor. This can be combined with previously described screening methods to isolate ligands exhibiting desired spectra of activities.

Expression in other cell types will often result in glycosylation differences in a particular chemokine or receptor. Various species variants may exhibit distinct functions based upon structural differences other than amino acid sequence. Differential modifications may be responsible for differential function, and elucidation of the effects are now made possible.

Thus, the present invention provides important reagents related to a physiological ligand-receptor interaction. Although the foregoing description has focused primarily upon the mouse and human embodiments of the chemokines or receptors specifically described, those of skill in the art will immediately recognize that the invention provides other species counterparts, e.g., rat and other mammalian species or allelic or polymorphic variants.

V. Antibodies

Antibodies can be raised to these chemokines or receptors, including species or polymorphic variants, and fragments thereof, both in their naturally occurring forms and in their recombinant forms. Additionally, antibodies can be raised to chemokines or receptors in either their active or inactive forms, or in their native or denatured forms. Anti-idiotypic antibodies are also contemplated.

Antibodies, including binding fragments and single chain versions, against predetermined fragments of the ligands can be raised by immunization of animals with concatemers or conjugates of the fragments with immunogenic proteins. Monoclonal antibodies are prepared from cells secreting the desired antibody. These antibodies can be screened for binding to normal or defective chemokines or receptors, or screened for agonistic or antagonistic activity. These monoclonal antibodies will usually bind with at least a K_{D} of about 1 mM, more usually at least about 300 μM, typically at least about 10 μM, more typically at least about 30 μM, preferably at least about 10 μM, and more preferably at least about 3 μM or better.

The antibodies, including antigen binding fragments, of this invention can have significant preparative, diagnostic, or therapeutic value. They can be useful to purify or label the desired antigen in a sample, or may be potent antagonists that bind to ligand and inhibit binding to receptor or inhibit the ability of a ligand to elicit a biological response. They also can be useful as non-neutralizing antibodies and can be coupled to, or as fusion proteins with, toxins or radionuclides so that when the antibody binds to antigen, a cell expressing it, e.g., on its surface via receptor, is killed. Further, these antibodies can be conjugated to drugs or other therapeutic agents, either directly or indirectly by means of a linker, and may effect drug targeting. Antibodies to receptors may be more easily used to block ligand binding and/or signal transduction.

The antibodies of this invention can also be useful in diagnostic or reagent purification applications. As capture or non-neutralizing antibodies, they can be screened for ability to bind to the chemokines or receptors without inhibiting ligand-receptor binding. As neutralizing antibodies, they can be useful in competitive binding assays. They will also be useful in detecting or quantifying chemokine or receptors, e.g., in immunoassays. They may be used as purification reagents in immunoaffinity columns or as immunohistochemistry reagents.

Ligand or receptor fragments may be concatenated or joined to other materials, particularly polypeptides, as fused or covalently joined polypeptides to be used as immunogens. Short peptides will preferably be made as repeat structures to increase size. A ligand and its fragments may be fused or covalently linked to a variety of immunogens, such as keyhole limpet hemocyanin, bovine serum albumin, tetanus toxoid, etc. See Microbiology, Hoeber Medical Division, Harper and Row, 1969; Landsteiner (1962) Specificity of Serological Reactions, Dover Publications, New York, and Williams, et al. (1967) Methods in Immunology and Immunochemistry, Vol. 1, Academic Press, New York, for descriptions of methods of preparing polyclonal antisera. A typical method involves hyperimmunization of an animal with an antigen. The blood of the animal is then collected shortly after the repeated immunizations and the gamma globulin fraction is isolated.

In some instances, it is desirable to prepare monoclonal antibodies from various mammalian hosts, such as mice, rodents, primates, humans, etc. Description of techniques for preparing such monoclonal antibodies may be found in, e.g., Stites, et al. (eds.) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed.) Academic Press, New York; and particularly in Kohler and Milstein (1975) in Nature 256:495-497, which discusses one method of generating monoclonal antibodies. Summarized briefly, this method involves injecting an animal with an immunogen. The animal is then sacrificed and cells taken, e.g., from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single cells from the immune animal generated in response to a specific site recognized on the immunogenic substance. Large amounts of antibody may be derived from ascites fluid from an animal.

Other suitable techniques involve in vitro exposure of lymphocytes to the antigenic polypeptides or alternatively to selection of libraries of antibodies in phage or similar vectors. See, Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents, teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly, U.S. Pat. No. 4,816,567; and Queen et al. (1989) Proc. Nat'l. Acad. Sci. 86:10029-10033.

The antibodies of this invention can also be used for affinity chromatography in isolating the protein. Columns can be prepared where the antibodies are linked to a solid support, e.g., particles, such as agarose, Sephadex, or the like, where a cell lysate may be passed through the column, the column washed, followed by increasing concentrations of a mild denaturant, whereby the purified chemokine protein will be released.

The antibodies may also be used to screen expression libraries for particular expression products. Usually the antibodies used in such a procedure will be labeled with a moiety allowing easy detection of presence of antigen by antibody binding.

Antibodies raised against these chemokines or receptors will also be useful to raise anti-idiotypic antibodies. These will be useful in detecting or diagnosing various immunological conditions related to expression of the respective antigens.

VI. Nucleic Acids

The described peptide sequences and the related reagents are useful in isolating a DNA clone encoding these chemokines or receptors, e.g., from a natural source. Typically, it will be useful in isolating a gene from another individual, and similar procedures will be applied to isolate genes from other species, e.g., warm blooded animals, such as birds and mammals. Cross hybridization will allow isolation of ligand from other species. A number of different approaches should be available to successfully isolate a suitable nucleic acid clone. Similar concepts apply to the receptor embodiments.

The purified protein or defined peptides are useful for generating antibodies by standard methods, as described above. Synthetic peptides or purified protein can be presented to an immune system to generate monoclonal or polyclonal antibodies. See, e.g., Coligan (1991) Current Protocols in Immunology Wiley/Greene; and Harlow and Lane (1989) Antibodies: A Laboratory Manual Cold Spring Harbor Press. Alternatively, a chemokine or receptor may be used as a specific binding reagent, and advantage can be taken of its specificity of binding, much like an antibody would be used. The chemokine receptors are typically 7 transmembrane proteins, which could be sensitive to appropriate interaction with lipid or membrane. The signal transduction typically is mediated through a G-protein, through interaction with a G-protein coupled receptor.

For example, the specific binding composition could be used for screening of an expression library made from a cell line which expresses a particular chemokine. The screening can be standard staining of surface expressed ligand, or by panning. Screening of intracellular expression can also be performed by various staining or immunofluorescence procedures. The binding compositions could be used to affinity purify or sort out cells expressing the ligand.

The peptide segments can also be used to predict appropriate oligonucleotides to screen a library, e.g., to isolate species variants. The genetic code can be used to select appropriate oligonucleotides useful as probes for screening. See, e.g., Tables 1 through 3. In combination with polymerase chain reaction (PCR) techniques, synthetic oligonucleotides will be useful in selecting correct clones from a library. Complementary sequences will also be used as probes or primers. Based upon identification of the likely amino terminus, the third peptide should be particularly useful, e.g., coupled with anchored vector or poly-A complementary PCR techniques or with complementary DNA of other peptides.

This invention contemplates use of isolated DNA or fragments to encode a biologically active corresponding chemokine polypeptide. In addition, this invention covers isolated or recombinant DNA which encodes a biologically active protein or polypeptide which is capable of hybridizing under appropriate conditions with the DNA sequences described herein. Said biologically active protein or polypeptide can be an intact ligand. receptor, or fragment, and have an amino acid sequence as disclosed in Tables 1 through 3. Further, this invention covers the use of isolated or recombinant DNA, or fragments thereof, which encode proteins which are homologous to a chemokine or receptor or which was isolated using such a cDNA encoding a chemokine or receptor as a probe. The isolated DNA can have the respective regulatory sequences in the 5′ and 3′ flanks, e.g., promoters, enhancers, poly-A addition signals, and others.

An “isolated” nucleic acid is a nucleic acid, e.g., an RNA, DNA, or a mixed polymer, which is substantially separated from other components which naturally accompany a native sequence, e.g., ribosomes, polymerases, and flanking genomic sequences from the originating species. The term embraces a nucleic acid sequence which has been removed from its naturally occurring environment, and includes recombinant or cloned DNA isolates and chemically synthesized analogs or analogs biologically synthesized by heterologous systems. A substantially pure molecule includes isolated forms of the molecule.

An isolated nucleic acid will generally be a homogeneous composition of molecules, but will, in some embodiments, contain minor heterogeneity. This heterogeneity is typically found at the polymer ends or portions not critical to a desired biological function or activity.

A “recombinant” nucleic acid is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring purified forms. Thus, for example, products made by transforming cells with any unnaturally occurring vector is encompassed, as are nucleic acids comprising sequence derived using a synthetic oligonucleotide process. Such is often done to replace a codon with a redundant codon encoding the same or a conservative amino acid, while typically introducing or removing a sequence recognition site. Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., fusion, polypeptide. Specifically included are synthetic nucleic acids which, by genetic code redundancy, encode polypeptides similar to fragments of these antigens, and fusions of sequences from various different species variants.

A significant “fragment” in a nucleic acid context is a contiguous segment of at least about 17 nucleotides, generally at least about 20 nucleotides, more generally at 35 least about 23 nucleotides, ordinarily at least about 26 nucleotides, more ordinarily at least about 29 nucleotides, often at least about 32 nucleotides, more often at least about 35 nucleotides, typically at least about 38 nucleotides, more typically at least about 41 nucleotides, usually at least about 44 nucleotides, more usually at least about 47 nucleotides, preferably at least about 50 nucleotides, more preferably at least about 53 nucleotides, and in particularly preferred embodiments will be at least about 56 or more nucleotides, e.g., 60, 65, 75, 85, 100, 120, 150, 200, 250, 300, 400, etc. Such fragments may have ends which begin and/or end at virtually all positions, e.g., beginning at nucleotides 1, 2, 3, etc., and ending at, e.g., 300, 299, 298, 287, etc., in combinatorial pairs. Particularly interesting polynucleotides have ends corresponding to structural domain boundaries.

A DNA which codes for a particular chemokine or receptor protein or peptide will be very useful to identify genes, mRNA, and cDNA species which code for related or homologous ligands or receptors, as well as DNAs which code for homologous proteins from different species. There are likely homologs in other species, including primates. Various chemokine proteins should be homologous and are encompassed herein, as would be receptors. However, even proteins that have a more distant evolutionary relationship to the ligands or receptors can readily be isolated under appropriate conditions using these sequences if they are sufficiently homologous. Primate chemokines or receptors are of particular interest.

This invention further covers recombinant DNA molecules and fragments having a DNA sequence identical to or highly homologous to the isolated DNAs set forth herein. In particular, the sequences will often be operably linked to DNA segments which control transcription, translation, and DNA replication. Alternatively, recombinant clones derived from the genomic sequences, e.g., containing introns, will be useful for transgenic studies, including, e.g., transgenic cells and organisms, and for gene therapy. See, e.g., Goodnow (1992) “Transgenic Animals” in Roitt (ed.) Encyclopedia of Immunology Academic Press, San Diego, pp. 1502-1504; Travis (1992) Science 256:1392-1394; Kuhn, et al. (1991) Science 254:707-710; Capecchi (1989) Science 244:1288; Robertson (1987)(ed.) Teratocarcinomas and Embryonic Stem Cells: A Practical Approach IRL Press, Oxford; and Rosenberg (1992) J. Clinical Oncology 10:180-199.

Homologous nucleic acid sequences, when compared, exhibit significant similarity, or identity. The standards for homology in nucleic acids are either measures for homology generally used in the art by sequence comparison or based upon hybridization conditions. The hybridization conditions are described in greater detail below.

Substantial homology in the nucleic acid sequence comparison context means either that the segments, or their complementary strands, when compared, are identical when optimally aligned, with appropriate nucleotide insertions or deletions, in at least about 50% of the nucleotides, generally at least about 56%, more generally at least about 59%, ordinarily at least about 62%, more ordinarily at least about 65%, often at least about 68%, more often at least about 71%, typically at least about 74%, more typically at least about 77%, usually at least about 80%, more usually at least about 85%, preferably at least about 90%, more preferably at least about 95 to 98% or more, and in particular embodiments, as high at about 99% or more of the nucleotides. Alternatively, substantial homology exists when the segments will hybridize under selective hybridization conditions, to a strand, or its complement, typically using a sequence derived from Tables 1 through 3. Typically, selective hybridization will occur when there is at least about 55% homology over a stretch of at least about 30 nucleotides, preferably at least about 65% over a stretch of at least about 25 nucleotides, more preferably at least about 75%, and most preferably at least about 90% over about 20 nucleotides. See, Kanehisa (1984) Nuc. Acids Res. 12:203-213. The length of homology comparison, as described, may be over longer stretches, and in certain embodiments will be over a stretch of at least about 17 nucleotides, usually at least about 20 nucleotides, more usually at least about 24 nucleotides, typically at least about 28 nucleotides, more typically at least about 40 nucleotides, preferably at least about 50 nucleotides, and more preferably at least about 75 to 100 or more nucleotides. PCR primers will generally have high levels of matches over potentially shorter lengths.

Stringent conditions, in referring to homology in the hybridization context, will be stringent combined conditions of salt, temperature, organic solvents, and other parameters, typically those controlled in hybridization reactions. Stringent temperature conditions will usually include temperatures in excess of about 30° C., more usually in excess of about 37° C., typically in excess of about 45° C., more typically in excess of about 55° C., preferably in excess of about 65° C., and more preferably in excess of about 70° C. Stringent salt conditions will ordinarily be less than about 1000 mM, usually less than about 500 mM, more usually less than about 400 mM, typically less than about 300 mM, preferably less than about 200 mM, and more preferably less than about 150 mM, e.g., 20-50 mM. However, the combination of parameters is much more important than the measure of any single parameter. See, e.g., Wetmur and Davidson (1968) J. Mol. Biol. 31:349-370.

Corresponding chemokines or receptors from other mammalian species can be cloned and isolated by cross-species hybridization of closely related species. Alternatively, sequences from a data base may be recognized as having similarity. Homology may be relatively low between distantly related species, and thus hybridization of relatively closely related species is advisable. Alternatively, preparation of an antibody preparation which exhibits less species specificity may be useful in expression cloning approaches. PCR approaches using segments of conserved sequences will also be used.

VII. Making Chemokines or Receptors; Mimetics

DNA which encodes each respective chemokine, receptor, or fragments thereof can be obtained by chemical synthesis, screening cDNA libraries, or by screening genomic libraries prepared from a wide variety of cell lines or tissue samples.

This DNA can be expressed in a wide variety of host cells for the synthesis of a full-length ligand or fragments which can in turn, for example, be used to generate polyclonal or monoclonal antibodies; for binding studies; for construction and expression of modified molecules; for expression cloning or purification; and for structure/function studies. Each antigen or its fragments can be expressed in host cells that are transformed or transfected with appropriate expression vectors. These molecules can be substantially purified to be free of protein or cellular contaminants, other than those derived from the recombinant host, and therefore are particularly useful in pharmaceutical compositions when combined with a pharmaceutically acceptable carrier and/or diluent. The antigens or antibodies, or portions thereof, may be expressed as fusions with other proteins.

Expression vectors are typically self-replicating DNA or RNA constructs containing the desired antigen gene or its fragments, usually operably linked to suitable genetic control elements that are recognized in a suitable host cell. These control elements are capable of effecting expression within a suitable host. The specific type of control elements necessary to effect expression will depend upon the eventual host cell used. Generally, the genetic control elements can include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also usually contain an origin of replication that allows the vector to replicate independently of the host cell.

The vectors of this invention contain DNA which encode embodiments of a chemokine, receptor, or a fragment thereof, typically encoding a biologically active polypeptide. The DNA can be under the control of a viral promoter and can encode a selection marker. This invention further contemplates use of such expression vectors which are capable of expressing eukaryotic cDNA coding for each chemokine or receptor in a prokaryotic or eukaryotic host, where the vector is compatible with the host and where the eukaryotic cDNA coding for the protein is inserted into the vector such that growth of the host containing the vector expresses the cDNA in question. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the ligand or its fragments in various hosts using vectors that do not contain a replication origin that is recognized by the host cell. It is also possible to use vectors that cause integration of a chemokine or receptor gene or its fragments into the host DNA by recombination, or to integrate a promoter which controls expression of an endogenous gene.

Vectors, as used herein, comprise plasmids, viruses, bacteriophage, integratable DNA fragments, and other vehicles, including those which enable the integration of DNA fragments into the genome of the host. Expression vectors are specialized vectors which contain genetic control elements that effect expression of operably linked genes. Plasmids are the most commonly used form of vector but many other forms of vectors which serve an equivalent function and which are, or become, known in the art are suitable for use herein. See, e.g., Pouwels, et al. (1985 and Supplements) Cloning Vectors: A Laboratory Manual, Elsevier, N.Y., and Rodriguez, et al. (1988)(eds.) Vectors: A Survey of Molecular Cloning Vectors and Their Uses, Buttersworth, Boston, Mass.

Transformed cells include cells, preferably mammalian, that have been transformed or transfected with a chemokine or receptor gene containing vector constructed using recombinant DNA techniques. Transformed host cells usually express the ligand, receptor, or its fragments, but for purposes of cloning, amplifying, and manipulating its DNA, do not need to express the protein. This invention further contemplates culturing transformed cells in a nutrient medium, thus permitting the protein to accumulate in the culture. The protein can be recovered, from the culture or from the culture medium, or from cell membranes.

For purposes of this invention, DNA sequences are operably linked when they are functionally related to each other. For example, DNA for a presequence or secretory signal is operably linked to a polypeptide if it is expressed as a preprotein or participates in directing the polypeptide to the cell membrane or in secretion of the polypeptide. A promoter is operably linked to a coding sequence if it controls the transcription of the polypeptide; a ribosome binding site is operably linked to a coding sequence if it is positioned to permit translation. Usually, operably linked means contiguous and in reading frame, however, certain genetic elements such as repressor genes are not contiguously linked but still bind to operator sequences that in turn control expression.

Suitable host cells include prokaryotes, lower eukaryotes, and higher eukaryotes. Prokaryotes include both gram negative and gram positive organisms, e.g., E. coli and B. subtilis. Lower eukaryotes include yeasts, e.g., S. cerevisiae and Pichia, and species of the genus Dictyostelium. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express these chemokines or their fragments include, but are not limited to, such vectors as those containing the lac promoter (pUC-series); trp promoter (pBR322-trp); Ipp promoter (the pIN-series); lambda-pP or pR promoters (pOTS); or hybrid promoters such as ptac (pDR540). See Brosius, et al. (1988) “Expression Vectors Employing Lambda-, trp-, lac-, and Ipp-derived Promoters”, in Rodriguez and Denhardt (eds.) Vectors: A Survey of Molecular Cloning Vectors and Their Uses, Buttersworth, Boston, Chapter 10, pp. 205-236.

Lower eukaryotes, e.g., yeasts and Dictyostelium, may be transformed with chemokine or receptor sequence containing nucleic acids. For purposes of this invention, the most common lower eukaryotic host is the baker's yeast, Saccharomyces cerevisiae. It will be used to generically represent lower eukaryotes although a number of other strains and species are also available. Yeast vectors typically consist of a replication origin (unless of the integrating type), a selection gene, a promoter, DNA encoding the desired protein or its fragments, and sequences for translation termination, polyadenylation, and transcription termination. Suitable expression vectors for yeast include such constitutive promoters as 3-phosphoglycerate kinase and various other glycolytic enzyme gene promoters or such inducible promoters as the alcohol dehydrogenase 2 promoter or metallothionine promoter. Suitable vectors include derivatives of the following types: self-replicating low copy number (such as the YRp-series), self-replicating high copy number (such as the YEp-series); integrating types (such as the YIp-series), or mini-chromosomes (such as the YCp-series).

Higher eukaryotic tissue culture cells are the preferred host cells for expression of the functionally active chemokine or receptor proteins. In principle, most any higher eukaryotic tissue culture cell line is workable, e.g., insect baculovirus expression systems, whether from an invertebrate or vertebrate source. However, mammalian cells are preferred, in that the processing, both cotranslationally and posttranslationally, will be typically most like natural. Transformation or transfection and propagation of such cells has become a routine procedure. Examples of useful cell lines include HeLa cells, Chinese hamster ovary (CHO) cell lines, baby rat kidney (BRK) cell lines, insect cell lines, bird cell lines, and monkey (COS) cell lines. Expression vectors for such cell lines usually include an origin of replication, a promoter, a translation initiation site, RNA splice sites (if genomic DNA is used), a polyadenylation site, and a transcription termination site. These vectors also usually contain a selection gene or amplification gene. Suitable expression vectors may be plasmids, viruses, or retroviruses carrying promoters derived, e.g., from such sources as from adenovirus, SV40, parvoviruses, vaccinia virus, or cytomegalovirus. Representative examples of suitable expression vectors include pCDNA1; pCD, see Okayama, et al. (1985) Mol. Cell Biol. 5:1136-1142; pMC1neo Poly-A, see Thomas, et al. (1987) Cell 51:503-512; and a baculovirus vector such as pAC 373 or pAC 610.

It will often be desired to express a chemokine or receptor polypeptide in a system which provides a specific or defined glycosylation pattern. In this case, the usual pattern will be that provided naturally by the expression system. However, the pattern will be modifiable by exposing the polypeptide, e.g., an unglycosylated form, to appropriate glycosylating proteins introduced into a heterologous expression system. For example, a chemokine or receptor gene may be co-transformed with one or more genes encoding mammalian or other glycosylating enzymes. Using this approach, certain mammalian glycosylation patterns will be achievable or approximated in prokaryote or other cells.

A chemokine, receptor, or a fragment thereof, may be engineered to be phosphatidyl inositol (PI) linked to a cell membrane, but can be removed from membranes by treatment with a phosphatidyl inositol cleaving enzyme, e.g., phosphatidyl inositol phospholipase-C. This releases the antigen in a biologically active form, and allows purification by standard procedures of protein chemistry. See, e.g., Low (1989) Biochim. Biophys. Acta 988:427-454; Tse, et al. (1985) Science 230:1003-1008; and Brunner, et al. (1991) J. Cell Biol. 114:1275-1283.

Now that these chemokines and receptors have been characterized, fragments or derivatives thereof can be prepared by conventional processes for synthesizing peptides. These include processes such as are described in Stewart and Young (1984) Solid Phase Peptide Synthesis, Pierce Chemical Co., Rockford, Ill.; Bodanszky and Bodanszky (1984) The Practice of Peptide Synthesis, Springer-Verlag, New York; and Bodanszky (1984) The Principles of Peptide Synthesis, Springer-Verlag, New York. For example, an azide process, an acid chloride process, an acid anhydride process, a mixed anhydride process, an active ester process (for example, p-nitrophenyl ester, N-hydroxysuccinimide ester, or cyanomethyl ester), a carbodiimidazole process, an oxidative-reductive process, or a dicyclohexylcarbodiimide (DCCD)/additive process can be used. Solid phase and solution phase syntheses are both applicable to the foregoing processes.

These chemokines, receptors, fragments, or derivatives are suitably prepared in accordance with the above processes as typically employed in peptide synthesis, generally either by a so-called stepwise process which comprises condensing an amino acid to the terminal amino acid, one by one in sequence, or by coupling peptide fragments to the terminal amino acid. Amino groups that are not being used in the coupling reaction are typically protected to prevent coupling at an incorrect location.

If a solid phase synthesis is adopted, the C-terminal amino acid is typically bound to an insoluble carrier or support through its carboxyl group. The insoluble carrier is not particularly limited as long as it has a binding capability to a reactive carboxyl group. Examples of such insoluble carriers include halomethyl resins, such as chloromethyl resin or bromomethyl resin, hydroxymethyl resins, phenol resins, tert-alkyloxycarbonyl-hydrazidated resins, and the like.

An amino group-protected amino acid is bound in sequence through condensation of its activated carboxyl group and the reactive amino group of the previously formed peptide or chain, to synthesize the peptide step by step. After synthesizing the complete sequence, the peptide is split off from the insoluble carrier to produce the peptide. This solid-phase approach is generally described, e.g., by Merrifield, et al. (1963) in J. Am. Chem. Soc. 85:2149-2156.

The prepared ligand and fragments thereof can be isolated and purified from the reaction mixture by means of peptide separation, e.g., by extraction, precipitation, electrophoresis, and various forms of chromatography, and the like. The various chemokines or receptors of this invention can be obtained in varying degrees of purity depending upon its desired use. Purification can be accomplished by use of the protein purification techniques disclosed herein or by the use of the antibodies herein described, e.g., in immunoabsorbant affinity chromatography. This immunoabsorbant affinity chromatography is typically carried out, e.g., by first linking the antibodies to a solid support and then contacting the linked antibodies with solubilized lysates of appropriate source cells, lysates of other cells expressing the ligand or receptor, or lysates or supernatants of cells producing the desired proteins as a result of DNA techniques, see below.

VIII. Uses

The present invention provides reagents which will find use in diagnostic applications as described elsewhere herein, e.g., in the general description for developmental abnormalities, or below in the description of kits for diagnosis.

This invention also provides reagents with significant therapeutic value. These chemokines and receptors (naturally occurring or recombinant), fragments thereof, and binding compositions, e.g., antibodies thereto, along with compounds identified as having binding affinity to them, should be useful in the treatment of conditions associated with abnormal physiology or development, including inflammatory conditions, e.g., asthma. In particular, modulation of trafficking of leukocytes is one likely biological activity, but a wider tissue distribution might suggest broader biological activity, including, e.g., antiviral effects. Abnormal proliferation, regeneration, degeneration, and atrophy may be modulated by appropriate therapeutic treatment using the compositions provided herein. For example, a disease or disorder associated with abnormal expression or abnormal signaling by a chemokine or ligand for a receptor should be a likely target for an agonist or antagonist of the ligand.

Various abnormal physiological or developmental conditions are known in cell types shown to possess the chemokine or receptor mRNAs by Northern blot analysis. See Berkow (ed.) The Merck Manual of Diagnosis and Therapy, Merck & Co., Rahway, N.J.; and Thorn, et al. Harrison's Principles of Internal Medicine, McGraw-Hill, N.Y. Developmental or functional abnormalities, e.g., of the immune system, cause significant medical abnormalities and conditions which may be susceptible to prevention or treatment using compositions provided herein.

Antibodies to the chemokines or receptors, including recombinant forms, can be purified and then used diagnostically or therapeutically, alone or in combination with other chemokines, cytokines, or antagonists thereof. These reagents can be combined for therapeutic use with additional active or inert ingredients, e.g., in conventional pharmaceutically acceptable carriers or diluents, e.g., immunogenic adjuvants, along with physiologically innocuous stabilizers and excipients. These combinations can be sterile filtered and placed into dosage forms as by lyophilization in dosage vials or storage in stabilized aqueous preparations. This invention also contemplates use of antibodies or binding fragments thereof, including forms which are not complement binding. Moreover, modifications to the antibody molecules or antigen binding fragments thereof, may be adopted which affect the pharmacokinetics or pharmacodynamics of the therapeutic entity.

Drug screening using antibodies or receptor or fragments thereof can be performed to identify compounds having binding affinity to each chemokine or receptor, including isolation of associated components. Subsequent biological assays can then be utilized to determine if the compound has intrinsic stimulating activity and is therefore a blocker or antagonist in that it blocks the activity of the ligand. Likewise, a compound having intrinsic stimulating activity can activate the receptor and is thus an agonist in that it simulates the activity of a ligand. This invention further contemplates the therapeutic use of antibodies to these chemokines as antagonists, or to the receptors as antagonists or agonists. This approach should be particularly useful with other chemokine or receptor species variants.

The quantities of reagents necessary for effective therapy will depend upon many different factors, including means of administration, target site, physiological state of the patient, and other medicants administered. Thus, treatment dosages should be titrated to optimize safety and efficacy in various populations, including racial subgroups, age, gender, etc. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa. Methods for administration are discussed therein and below, e.g., for oral, intravenous, intraperitoneal, or intramuscular administration, transdermal diffusion, and others. Pharmaceutically acceptable carriers typically include water, saline, buffers, and other compounds described, e.g., in the Merck Index, Merck & Co., Rahway, N.J. Dosage ranges would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Slow release formulations, or a slow release apparatus will often be utilized for continuous administration.

A chemokine, fragments thereof, or antibodies to it or its fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in many conventional dosage formulations. While it is possible for the active ingredient to be administered alone, it is often preferable to present it as a pharmaceutical formulation. Formulations typically comprise at least one active ingredient, as defined above, together with one or more acceptable carriers thereof. Each carrier should be both pharmaceutically and physiologically acceptable in the sense of being compatible with the other ingredients and not injurious to the patient. Carriers may improve storage life, stability, etc. Formulations include those suitable for oral, rectal, nasal, or parenteral (including subcutaneous, intramuscular, intravenous and intradermal) administration. The formulations may conveniently be presented in unit dosage form and may be prepared by any methods well known in the art of pharmacy. See, e.g., Gilman, et al. (eds.) (1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds.) (1993) Pharmaceutical Dosage Forms: Parenteral Medications Dekker, N.Y.; Lieberman, et al. (eds.) (1990) Pharmaceutical Dosage Forms: Tablets Dekker, N.Y.; and Lieberman, et al. (eds.) (1990) Pharmaceutical Dosage Forms: Disperse Systems Dekker, N.Y. The therapy of this invention may be combined with or used in association with other therapeutic agents. Similar considerations will often apply to receptor based reagents.

Both the naturally occurring and the recombinant forms of the chemokines or receptors of this invention are particularly useful in kits and assay methods which are capable of screening compounds for binding activity to the proteins. Several methods of automating assays have been developed in recent years so as to permit screening of tens of thousands of compounds in a short period. See, e.g., Fodor, et al. (1991) Science 251:767-773, which describes means for testing of binding affinity by a plurality of defined polymers synthesized on a solid substrate. The development of suitable assays can be greatly facilitated by the availability of large amounts of purified, soluble chemokine as provided by this invention.

For example, antagonists can normally be found once a ligand has been structurally defined. Testing of potential ligand analogs is now possible upon the development of highly automated assay methods using physiologically responsive cells. In particular, new agonists and antagonists will be discovered by using screening techniques described herein.

Viable cells could also be used to screen for the effects of drugs on respective chemokine or G-protein coupled receptor mediated functions, e.g., second messenger levels, i.e., Ca⁺⁺; inositol phosphate pool changes (see, e.g., Berridge (1993) Nature 361:315-325 or Billah and Anthes (1990) Biochem. J. 269:281-291); cellular morphology modification responses; phosphoinositide lipid turnover; an antiviral response, and others. Some detection methods allow for elimination of a separation step, e.g., a proximity sensitive detection system. Calcium sensitive dyes will be useful for detecting Ca⁺⁺ levels, with a fluorimeter or a fluorescence cell sorting apparatus.

Rational drug design may also be based upon structural studies of the molecular shapes of the chemokines, other effectors or analogs, or the receptors. Effectors may be other proteins which mediate other functions in response to ligand binding, or other proteins which normally interact with the receptor. One means for determining which sites interact with specific other proteins is a physical structure determination, e.g., x-ray crystallography or 2 dimensional NMR techniques. These will provide guidance as to which amino acid residues form molecular contact regions. For a detailed description of protein structural determination, see, e.g., Blundell and Johnson (1976) Protein Crystallography, Academic Press, New York.

Purified chemokine or receptor can be coated directly onto plates for use in the aforementioned drug screening techniques, and may be associated with detergents or lipids. However, non-neutralizing antibodies, e.g., to the chemokines or receptors can be used as capture antibodies to immobilize the respective protein on the solid phase.

Similar concepts also apply to the chemokine receptor embodiments of the invention.

IX. Kits

This invention also contemplates use of chemokine or receptor proteins, fragments thereof, peptides, binding compositions, and their fusion products in a variety of diagnostic kits and methods for detecting the presence of ligand, antibodies, or receptors. Typically the kit will have a compartment containing a defined chemokine or receptor peptide or gene segment or a reagent which recognizes one or the other, e.g., binding reagents.

A kit for determining the binding affinity of a test compound to a chemokine or receptor would typically comprise a test compound; a labeled compound, for example an antibody having known binding affinity for the protein; a source of chemokine or receptor (naturally occurring or recombinant); and a means for separating bound from free labeled compound, such as a solid phase for immobilizing the ligand or receptor. Once compounds are screened, those having suitable binding affinity to the ligand or receptor can be evaluated in suitable biological assays, as are well known in the art, to determine whether they act as agonists or antagonists to the receptor. The availability of recombinant chemokine or receptor polypeptides also provide well defined standards for calibrating such assays or as positive control samples.

A preferred kit for determining the concentration of, for example, a chemokine or receptor in a sample would typically comprise a labeled compound, e.g., antibody, having known binding affinity for the target, a source of ligand or receptor (naturally occurring or recombinant) and a means for separating the bound from free labeled compound, for example, a solid phase for immobilizing the chemokine or receptor. Compartments containing reagents, and instructions for use or disposal, will normally be provided.

Antibodies, including antigen binding fragments, specific for the chemokine or receptor, or fragments are useful in diagnostic applications to detect the presence of elevated levels of chemokine, receptor, and/or its fragments. Such diagnostic assays can employ lysates, live cells, fixed cells, immunofluorescence, cell cultures, body fluids, and further can involve the detection of antigens related to the ligand or receptor in serum, or the like. Diagnostic assays may be homogeneous (without a separation step between free reagent and antigen complex) or heterogeneous (with a separation step). Various commercial assays exist, such as radioimmunoassay (RIA), enzyme-linked immunosorbent assay (ELISA), enzyme immunoassay (EIA), enzyme-multiplied immunoassay technique (EMIT), substrate-labeled fluorescent immunoassay (SLFIA), and the like. For example, unlabeled antibodies can be employed by using a second antibody which is labeled and which recognizes the primary antibody to a chemokine or receptor or to a particular fragment thereof. Similar assays have also been extensively discussed in the literature. See, e.g., Harlow and Lane (1988) Antibodies: A Laboratory Manual, CSH.

Anti-idiotypic antibodies may have similar uses to diagnose presence of antibodies against a chemokine or receptor, as such may be diagnostic of various abnormal states. For example, overproduction of a chemokine or receptor may result in production of various immunological reactions which may be diagnostic of abnormal physiological states, particularly in various inflammatory or asthma conditions.

Frequently, the reagents for diagnostic assays are supplied in kits, so as to optimize the sensitivity of the assay. For the subject invention, depending upon the nature of the assay, the protocol, and the label, either labeled or unlabeled antibody or labeled chemokine or receptor is provided. This is usually in conjunction with other additives, such as buffers, stabilizers, materials necessary for signal production such as substrates for enzymes, and the like. Preferably, the kit will also contain instructions for proper use and disposal of the contents after use. Typically the kit has compartments for each useful reagent. Desirably, the reagents are provided as a dry lyophilized powder, where the reagents may be reconstituted in an aqueous medium providing appropriate concentrations of reagents for performing the assay.

The aforementioned constituents of the drug screening and the diagnostic assays may be used without modification or may be modified in a variety of ways. For example, labeling may be achieved by covalently or non-covalently joining a moiety which directly or indirectly provides a detectable signal. In any of these assays, the ligand, test compound, chemokine, receptor, or antibodies thereto can be labeled either directly or indirectly. Possibilities for direct labeling include label groups: radiolabels such as ¹²⁵I, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups.

There are also numerous methods of separating bound from the free ligand, or alternatively bound from free test compound. The chemokine or receptor can be immobilized on various matrixes, perhaps with detergents or associated lipids, followed by washing. Suitable matrixes include plastic such as an ELISA plate, filters, and beads. Methods of immobilizing the chemokine or receptor to a matrix include, without limitation, direct adhesion to plastic, use of a capture antibody, chemical coupling, and biotin-avidin. The last step in this approach may involve the precipitation of antigen/antibody complex by any of several methods including those utilizing, e.g., an organic solvent such as polyethylene glycol or a salt such as ammonium sulfate. Other suitable separation techniques include, without limitation, the fluorescein antibody magnetizable particle method described in Rattle, et al. (1984) Clin. Chem. 30:1457-1461, and the double antibody magnetic particle separation as described in U.S. Pat. No. 4,659,678.

Methods for linking proteins or their fragments to the various labels have been extensively reported in the literature and do not require detailed discussion here. Many of the techniques involve the use of activated carboxyl groups either through the use of carbodiimide or active esters to form peptide bonds, the formation of thioethers by reaction of a mercapto group with an activated halogen such as chloroacetyl, or an activated olefin such as maleimide, for linkage, or the like. Fusion proteins will also find use in these applications.

Another diagnostic aspect of this invention involves use of oligonucleotide or polynucleotide sequences taken from the sequence of the chemokine or receptor. These sequences can be used as probes for detecting levels of the ligand message in samples from patients suspected of having an abnormal condition, e.g., an inflammatory, physiological, or developmental problem. The preparation of both RNA and DNA nucleotide sequences, the labeling of the sequences, and the preferred size of the sequences has received ample description and discussion in the literature. Normally an oligonucleotide probe should have at least about 14 nucleotides, usually at least about 18 nucleotides, and the polynucleotide probes may be up to several kilobases. Various labels may be employed, most commonly radionuclides, particularly ³²P. However, other techniques may also be employed, such as using biotin modified nucleotides for introduction into a polynucleotide. The biotin then serves as the site for binding to avidin or antibodies, which may be labeled with a wide variety of labels, such as radionuclides, fluorescers, enzymes, or the like. Alternatively, antibodies may be employed which can recognize specific duplexes, including DNA duplexes, RNA duplexes, DNA-RNA hybrid duplexes, or DNA-protein duplexes. The antibodies in turn may be labeled and the assay carried out where the duplex is bound to a surface, so that upon the formation of duplex on the surface, the presence of antibody bound to the duplex can be detected. The use of probes to the novel anti-sense RNA may be carried out in conventional techniques such as nucleic acid hybridization, plus and minus screening, recombinational probing, hybrid released translation (HRT), and hybrid arrested translation (HART). This also includes amplification techniques such as polymerase chain reaction (PCR).

Diagnostic kits which also test for the qualitative or quantitative presence of other markers are also contemplated. Diagnosis or prognosis may depend on the combination of multiple indications used as markers. Thus, kits may test for combinations of markers. See, e.g., Viallet, et al. (1989) Progress in Growth Factor Res. 1:89-97.

X. Receptor for Chemokine; Ligands for Receptors

Having isolated a ligand binding partner of a specific interaction, methods exist for isolating the counter-partner. See, Gearing, et al EMBO J. 8:3667-4676 or McMahan, et al. (1991) EMBO J. 10:2821-2832. For example, means to label a chemokine without interfering with the binding to its receptor can be determined. For example, an affinity label can be fused to either the amino- or carboxy-terminus of the ligand. An expression library can be screened for specific binding of chemokine, e.g., by cell sorting, or other screening to detect subpopulations which express such a binding component. See, e.g., Ho, et al. (1993) Proc. Nat'l Acad. Sci. 90:11267-11271. Alternatively, a panning method may be used. See, e.g., Seed and Aruffo (1987) Proc. Nat'l. Acad. Sci. 84:3365-3369.

With a receptor, means to identify the ligand exist. Methods for using the receptor, e.g., on the cell membrane, can be used to screen for ligand by, e.g., assaying for a common G-protein linked signal such as Ca++ flux. See Lerner (1994) Trends in Neurosciences 17:142-146. It is likely that the ligands for these receptors are chemokines.

Protein cross-linking techniques with label can be applied to a isolate binding partners of a chemokine. This would allow identification of protein which specifically interacts with a chemokine, e.g., in a ligand-receptor like manner.

The broad scope of this invention is best understood with reference to the following examples, which are not intended to limit the invention to specific embodiments.

### EXAMPLES

I. General Methods

Some of the standard methods are described or referenced, e.g., in Maniatis, et al. (1982) Molecular Cloning. A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, NY; Ausubel, et al., Biology Greene Publishing Associates, Brooklyn, N.Y.; or Ausubel, et al. (1987 and Supplements) Current Protocols in Molecular Biology Greene/Wiley, New York; Innis, et al. (eds.)(1990) PCR Protocols: A Guide to Methods and Applications Academic Press, N.Y. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering. Principle and Methods 12:87-98, Plenum Press, N.Y.; and Crowe, et al. (1992) OIAexpress: The High Level Expression & Protein Purification System QUIAGEN, Inc., Chatsworth, Calif.

FACS analyses are described in Melamed, et al. (1990) Flow Cytometry and Sorting Wiley-Liss, Inc., New York, N.Y.; Shapiro (1988) Practical Flow Cytometry Liss, New York, N.Y.; and Robinson, et al. (1993) Handbook of Flow Cytometry Methods Wiley-Liss, New York, N.Y.

II. Isolation and Characterization of Chemokine cDNA

The CKDLR20.1 was isolated from a cDNA library made from the lung from a RAG-1 “knockout” mouse. See, Mombaerts, et al. (1992) Cell 68:869-877. A cDNA probe which comprises the entire coding portion of human MIP-3a (see Gish, et al., U.S. Ser. No. 08/675,814) was used as a probe. This identified a gene designated CKDLR20.1, which is characterized in Table 1. Individual cDNA clones were sequenced using standard methods, e.g., the Taq DyeDeoxy Terminator Cycle Sequencing kit (Applied Biosystems, Foster City, Calif.), and the sequence was further characterized.

The predicted signal sequence corresponds to amino acids 1 (met) to about 18 (thr), so the mature form should begin with gln and run about 142 amino acids. Additional processing may occur in a physiological system. The message is upregulated in Nippostrongylus brasilensis parasite infected lung tissue, and its mRNA expression appears fairly restricted to lung tissue. But such may suggest a role in other mucosal boundaries, e.g., skin or gut.

Computer analysis for related genes indicates the closest match is to human IL-8, for which no mouse counterpart has yet been identified. However, the gene exhibits significant homology at the amino acid levels with other CXC chemokine family members. The existence of the ELR sequence immediately upstream from the CXC motif suggests that the chemokine has a pro-inflammatory activity.

A human counterpart should be isolatable using the entire coding portion of the mouse clone as a hybridization probe. A Southern blot may indicate the extent of homology across species, and either a cDNA library or mRNA can be screened to identify an appropriate cell source. The physiological state of many different cell types may also be evaluated for increased expression of the gene.

III. Isolation and Characterization of GPCR cDNAs

A. 69A08 from Mouse

The mouse 69A08 clone was derived from mouse pre-T cells. The nucleotide sequence is provided in Table 2, encoding a polypeptide of about 359 amino acids.

Computer analysis suggests that the closest related genes are orphan G-protein coupled receptors. These include the chemokine receptors, and protease, e.g., thrombin, receptors. Structural motifs suggest that the receptor contains motifs characteristic of the chemokine receptor family, and of the protease receptor family. The transmembrane segments, based upon hydrophobicity plots and comparisons with other similar GPCRs, should be about as follows (SEQ ID NO: 6): TM1 from 57 (ala) to 74 (leu); TM2 from 93 (leu) to 109 (ala); TM3 from 122 (ala) to 147 (leu); TM4 from 167 (leu) to 189 (his), but with the hydrophobic region reaching as far as 205 (met); TM5 from 222 (ala) to 248 (ala); TM6 from 256 (ala) to 281 (his); and TM7 from 293 (leu) to 318 (val). See, e.g., Loetscher, et al. (1996) J. Expt'l Med. 184:963-969. A DRY motif is found, e.g., near residue 149. The amino terminal segment is probably an extracellular segment (E1), and the others would be E2 between TM2 and TM3; E3 between TM4 and TM5; and E4 between TM6 and TM7. The intracellular segments should then run I1 between TM1 and TM2; I2 between TM3 and TM4, I3 between TM5 and TM6, and I4 the carboxy terminus from the end of TM7. Additional processing may occur in a physiological system.

B. HSD12 from Human

The human HSD12 clone was derived from a cDNA library made from human monocytes or dendritic cells. Individual cDNA clones are sequenced using standard methods, and the sequence was identified and further characterized. The nucleotide sequence is provided in Table 3, encoding a polypeptide of about 371 amino acids. The natural message appears to be about 2.8 kB, which contains a poly-A tail. The message contains an Alu repeat in the region of about 2450-2825.

Computer analysis suggests that the closest related genes are orphan G-protein coupled receptors. The transmembrane segments, based upon hydrophobicity plots and comparisons with other similar GPCRs, should be about as follows (SEQ ID NO: 8): TM1 from 34 (ile) to 50 (ala); TM2 from 67 (val) to 83 (thr); TM3 from 112 (ile) to 128 (ser); TM4 from 147 (arg) to 165 (his); TM5 from 193 (ala) to 209 (thr); TM6 from 238 (val) to 254 (leu); and TM7 from 282 (val) to 298 (ile). The amino terminal segment is probably an extracellular segment (E1), and the others would be E2 between TM2 and TM3; E3 between TM4 and TM5; and E4 between TM6 and TM7. The cytoplasmic, or intracellular, segments should then run C1between TM1 and TM2; C2 between TM3 and TM4, C3 between TM5 and TM6, and C4 the carboxy terminus from the end of TM7. Additional processing may occur in a physiological system. Of particular importance in these receptors are the C3 segment, which is usually the longest of the cytoplasmic segments, and which probably provides specificity for binding of signaling components, e.g., the G proteins.

IV. Preparation of Antibodies

Many standard methods are available for preparation of antibodies. For example, synthetic peptides may be prepared to be used as antigen, administered to an appropriate animal, and either polyclonal or monoclonal antibodies prepared. Short peptides, e.g., less than about 10 amino acids may be expressed as repeated sequences, while longer peptides may be used alone or conjugated to a carrier. For example, with the GPCRs, animals are immunized with peptides or complete proteins from Tables 2 or 3. Highest specificity will result when the polypeptides are selected from portions which are most unique, e.g., not from conserved sequence regions. The animals may be used to collect antiserum, or may be used to generate monoclonal antibodies.

Antiserum is evaluated for use, e.g., in an ELISA, and will be evaluated for utility in immunoprecipitation, e.g., typically native, or Western blot, e.g., denatured antigen, analysis. Monoclonal antibodies will also be evaluated for those same uses.

The antibodies provided will be useful as immunoaffinity reagents, as detection reagents, for immunohistochemistry, and as potential therapeutic reagents, either as agonist or antagonist reagents. They will often be in sterile formulations.

V. Assays for Chemotactic Activity of Chemokines

Chemokine proteins are produced, e.g., in COS cells transfected with a plasmid carrying the chemokine cDNA by electroporation. See, Hara, et al. (1992) EMBO J. 10:1875-1884. Physical analytical methods may be applied, e.g., CD analysis, to compare tertiary structure to other chemokines to evaluate whether the protein has likely folded into an active conformation. After transfection, a culture supernatant is collected and subjected to bioassays. A mock control, e.g., a plasmid carrying the luciferase cDNA, is used. See, de Wet, et al. (1987) Mol. Cell. Biol. 7:725-757. A positive control, e.g., recombinant murine MIP-1α from R&D Systems (Minneapolis, Minn.), is typically used. Likewise, antibodies may be used to block the biological activities, e.g., as a control.

Lymphocyte migration assays are performed as previously described, e.g., in Bacon, et al. (1988) Br. J. Pharmacol. 95:966-974. Murine Th2 T cell clones, CDC-25 (see Tony, et al. (1985) J. Exp. Med. 161:223-241) and HDK-1 (see Cherwinski, et al. (1987) J. Exp. Med. 166:1229-1244), made available from R. Coffman and A. O'Garra (DNAX, Palo Alto, Calif.), respectively, are used as controls.

Ca2+ flux upon chemokine stimulation is measured, e.g., according to the published procedure described in Bacon, et al. (1995) J. Immunol. 154:3654-3666.

Maximal numbers of migrating cells in response to the CKDLR20.1 are measured. See Schall (1993) J. Exp. Med. 177:1821-1826. A dose-response curve is determined, preferably giving a characteristic bell shaped dose-response curve.

After stimulation with various chemokines, lymphocytes often exhibit a measurable intracellular Ca2+ flux. MIP-1α, e.g., is capable of inducing immediate transients of calcium mobilization. Typically, the levels of chemokine used in these assays will be comparable to those used for the chemotaxis assays (1/1000 dilution of conditioned supernatants).

Retroviral infection assays have also been described, and recent description of certain chemokine receptors in retroviral infection processes may indicate that similar roles may apply these receptors. See, e.g., Balter (1996) Science 272:1740 (describing evidence for chemokine receptors as coreceptors for HIV); and Deng, et al. (1996) Nature 381:661-666.

For receptors, biological activity may be measured in response to an appropriate ligand. The receptors are transfected into an assortment of cell types, each of which is likely to possess the intracellular signaling components compatible with the expressed receptor. Various ligand sources are tested to find a source of ligand which results in a G-protein coupled response. Alternatively, the cells are tested for Ca++ flux in response to such ligands. Flux may be conveniently measured by electrophysiological means, or by Ca++ sensitive dyes.

VI. Expression Analysis of Chemokine/Receptor Genes

RNA blot and hybridization are performed according to the standard methods in Maniatis, et al. (1982) Molecular Cloning: A Laboratory Manual Cold Spring Harbor Laboratory Press, Cold Spring Harbor, N.Y. An appropriate fragment or the whole coding sequence of a cDNA fragment is selected for use as a probe. To verify the amount of RNA loaded in each lane, the substrate membrane is reprobed with a control cDNA, e.g., glyceraldehyde 3-phosphate dehydrogenase (G3PDH) cDNA (Clontech, Palo Alto Calif.).

Analysis of mRNA from the appropriate cell source using the probe will determine the natural size of message. It will also indicate whether different sized messages exist. The messages will be subject to analysis after isolation, e.g., by PCR or hybridization techniques.

Northern blot analysis may be performed on many different mRNA sources, e.g., different tissues, different species, or cells exhibiting defined physiological responses, e.g., activation conditions or developmental conditions. However, in certain cases, cDNA libraries may be used to evaluate sources which are difficult to prepare. A “reverse Northern” uses cDNA inserts removed from vector, but multiplicity of bands may reflect either different sized messages, or may be artifact due to incomplete reverse transcription in the preparation of the cDNA library. In such instances, verification may be appropriate by standard Northern analysis.

Similarly, Southern blots may be used to evaluate species distribution of a gene. The stringency of washes of the blot will also provide information as to the extent of homology of various species counterparts.

Tissue distribution, and cell distribution, may be evaluated by immunohistochemistry using antibodies. Alternatively, in situ nucleic acid hybridization may also be used in such analysis.

A. CKDLR20.1

The CKDLR20.1 was isolated from a RAG-1 “knockout” mouse. Several cell lines were tested for expression using Northern blot technology, and were found negative. These cell lines included bone marrow stroma (3D1), mast cells (MC9), αβ CD4- CD8- hybridoma (A3.2), T cell clone (HT-2), fibroblast (L cell), pro-T hybridoma, pre-T hybridoma, B cell (A20-2J) and CD3- CD4- CD8- (BW) cells. The expression level was high in lung, with weak signals in fetal lung and heart, and no detectable signal in fetal liver, thymus, activated spleen, lymph node, brain, or kidney.

The expression pattern might suggest that the pro-inflammatory chemokine may be involved various aspects of the lung physiology, e.g., the initiation or maintenance of an asthmatic condition. It may play a role in pneumonia, or in various occupational lung conditions, e.g., black lung, farmer's lung, silicosis, asbestosis, or various hypersensitivity lung conditions. See, e.g., Berkow (ed.) The Merck Manual of Diagnosis and Therapy, Merck & Co., Rahway, N.J.; and Thorn, et al. Harrison's Principles of Internal Medicine, McGraw-Hill, N.Y.

The ELR motif may also suggest a role in angiogenesis, which may suggest that antagonists, or possibly agonists in other situations, may be useful in treating lung or other tumors, e.g., of various mucosal surfaces such as the gut or skin. It may also be useful in treatment of lung neoplastic conditions, e.g., lung cancers.

The combination of the structure and distribution of this chemokine suggests a role in lung physiology, and perhaps general mucosal immunity.

Samples for mouse mRNA isolation may include, e.g.: resting mouse fibroblastic L cell line (C200); Braf:ER (Braf fusion to estrogen receptor) transfected cells, control (C201); T cells, TH1 polarized (Mell4 bright, CD4+ cells from spleen, polarized for 7 days with IFN-γ and anti IL-4; T200); T cells, TH2 polarized (Mell4 bright, CD4+ cells from spleen, polarized for 7 days with IL-4 and anti-IFN-γ; T201); T cells, highly TH1 polarized (see Openshaw, et al. (1995) J. Exp. Med. 182:1357-1367; activated with anti-CD3 for 2, 6, 16 h pooled; T202); T cells, highly TH2 polarized (see Openshaw, et al. (1995) J. Exp. Med. 182:1357-1367; activated with anti-CD3 for 2, 6, 16 h pooled; T203); CD44− CD25+ pre T cells, sorted from thymus (T204); TH1 T cell clone D1.1, resting for 3 weeks after last stimulation with antigen (T205); TH1 T cell clone D1.1, 10 μg/ml ConA stimulated 15 h (T206); TH2 T cell clone CDC35, resting for 3 weeks after last stimulation with antigen (T207); TH2 T cell clone CDC35, 10 μg/ml ConA stimulated 15 h (T208); Mell4+ naive T cells from spleen, resting (T209); Mell4+ T cells, polarized to Th1 with IFN-γ/IL-12/anti-IL-4 for 6, 12, 24 h pooled (T210); Mell4+ T cells, polarized to Th2 with IL-4/anti-IFN-γ for 6, 13, 24 h pooled (T211); unstimulated mature B cell leukemia cell line A20 (B200); unstimulated B cell line CH12 (B201); unstimulated large B cells from spleen (B202); B cells from total spleen, LPS activated (B203); metrizamide enriched dendritic cells from spleen, resting (D200); dendritic cells from bone marrow, resting (D201); monocyte cell line RAW 264.7 activated with LPS 4 h (M200); bone-marrow macrophages derived with GM and M-CSF (M201); macrophage cell line J774, resting (M202); macrophage cell line J774+LPS+anti-IL-10 at 0.5, 1, 3, 6, 12 h pooled (M203); macrophage cell line J774+LPS+IL-10 at 0.5, 1, 3, 5, 12 h pooled(M204); aerosol challenged mouse lung tissue, Th2 primers, aerosol OVA challenge 7, 14, 23 h pooled (see Garlisi, et al. (1995) Clinical Immunology and Immunopathology 75:75-83; X206); Nippostrongulus-infected lung tissue (see Coffman, et al. (1989) Science 245:308-310; X200); total adult lung, normal (O200); total lung, rag-1 (see Schwarz, et al. (1993) Immunodeficiency 4:249-252; O205); IL-10 K.O. spleen (see Kuhn, et al. (1991) Cell 75:263-274; X201); total adult spleen, normal (O201); total spleen, rag-1 (O207); IL-10 K.O. Peyer's patches (O202); total Peyer's patches, normal (O210); IL-10 K.O. mesenteric lymph nodes (X203); total mesenteric lymph nodes, normal (O211); IL-10 K.O. colon (X203); total colon, normal (O212); NOD mouse pancreas (see Makino, et al. (1980) Jikken Dobutsu 29:1-13; X205); total thymus, rag-1 (O208); total kidney, rag-1 (O209); total heart, rag-1 (O202); total brain, rag-1 (O203); total testes, rag-1 (O204); total liver, rag-1 (O206); rat normal joint tissue (O300); and rat arthritic joint tissue (X300).

B. 69A08

The 69A08 gene was identified from a cDNA library made from thymus pre-T cells. Hybridization analysis detected a positive mRNA signal in activated T cells, Th1 and Th2 cell libraries, macrophages, and tissue prepared from mice infected with Nippostrongylus brasiliensis. This suggests a role of the molecule in the immune response, e.g., inflammation or vascular biology. The molecule, or its antagonist, should be useful in various inflammatory disease states or conditions, e.g., in the lung or elsewhere, including skin and gut.

C. HSD12

Southern Analysis: DNA (5 μg) from a primary amplified cDNA library was digested with appropriate restriction enzymes to release the inserts, run on a 1% agarose gel and transferred to a nylon membrane (Schleicher and Schuell, Keene, N.H.).

Samples for human mRNA isolation include, e.g.: U937 premonocytic line, resting (M100); elutriated monocytes, activated with LPS, IFNγ, anti-IL-10 for 1, 2, 6, 12, 24 h pooled (M102); elutriated monocytes, activated with LPS, IFNγ, IL-10 for 1, 2, 6, 12, 24 h pooled (M103); elutriated monocytes, activated LPS for 1 h (M108); elutriated monocytes, activated LPS for 6 h (M109); DC 70% CD1a+, from CD34+ GM-CSF, TNFα 12 days, resting (D101); DC 70% CD1a+, from CD34+ GM-CSF, TNFα 12 days, activated with PMA and ionomycin for 1 hr (D102); DC 70% CD1a+, from CD34+ GM-CSF, TNFα 12 days, activated with PMA and ionomycin for 6 hr (D103); DC from monocytes GM-CSF, IL-4 5 days, activated LPS 4, 16 h pooled (D109); DC from monocytes GM-CSF, IL-4 5 days, activated TNFα, monocyte supe for 4, 16 h pooled (D110); peripheral blood mononuclear cells (monocytes, T cells, NK cells, granulocytes, B cells), resting (T100); peripheral blood mononuclear cells, activated with anti-CD3 for 2, 6, 12 h pooled (T101); T cell, TH1 clone HY06, resting (T107); T cell, TH1 clone HY06, activated with anti-CD28 and anti-CD3 for 3, 6, 12 h pooled (T108); T cell, TH1 clone HY06, anergic treated with specific peptide for 2, 6, 12 h pooled (T109); T cell, TH2 clone HY935, resting (T110); T cell, TH2 clone HY935, activated with anti-CD28 and anti-CD3 for 2, 7, 12 h pooled (T111); T cell, TH2 clone HY935, resting (T110); T cell, TH2 clone HY935, activated with anti-CD28 and anti-CD3 for 2, 7, 12 h pooled (T111); B cell EBV lines pooled WT49, RSB, JY, CVIR, 721.221, RM3, HSY, resting (B102); splenocytes, resting (B100); splenocytes, activated with anti-CD40 and IL-4 (B101); NK 20 clones pooled, resting (K100); NK 20 clones pooled, activated with PMA and ionomycin for 6 h (K101); kidney fetal 28 wk male (O100); lung fetal 28 wk male (O101); liver fetal 28 wk male (O102); heart fetal 28 wk male (O103); brain fetal 28 wk male (O104); gallbladder fetal 28 wk male (O106); small intestine fetal 28 wk male (O107); adipose tissue fetal 28 wk male (O108); ovary fetal 25 wk female (O109); testes fetal 28 wk male (O111); uterus fetal 25 wk female (O110); spleen fetal 28 wk male (O112); adult placenta 28 wk (O113); and mouse monocyte cell line RAW 264.7 activated with LPS 4 h (M200).

The HSD12 gene was identified from a cDNA library made from activated dendritic cells. It has also been detected by hybridization in monocytes and dendritic cells, with lower signals detected in Th1 cells and NK cells. Dendritic cells derived from CD34+ cells seem to express more than those which are monocyte derived. The expression levels seem lower in either resting or anergic cell libraries. In the NK cells, the activated cells had higher expression levels than resting. The expression in dendritic cells suggests a role in immune function, e.g., where dendritic cells are important. Thus includes antigen presentation, and initiation of an immune response. Thus, agonists or antagonists of the receptor should be useful in such immune functions.

VII. Screening for Receptor/Ligand

Labeled reagent is useful for screening of an expression library made from a cell line which expresses a chemokine or receptor, as appropriate. Standard staining techniques are used to detect or sort intracellular or surface expressed ligand, or surface expressing transformed cells are screened by panning. Screening of intracellular expression is performed by various staining or immunofluorescence procedures. See also, e.g., McMahan, et al. (1991) EMBO J. 10:2821-2832.

For example, on day 0, precoat 2-chamber permanox slides with 1 ml per chamber of fibronectin, 10 ng/ml in PBS, for 30 min at room temperature. Rinse once with PBS. Then plate COS cells at 2-3×10⁵ cells per chamber in 1.5 ml of growth media. Incubate overnight at 37° C.

On day 1 for each sample, prepare 0.5 ml of a solution of 66 μg/ml DEAE-dextran, 66 μM chloroquine, and 4 μg DNA in serum free DME. For each set, a positive control is prepared, e.g., of huIL-10-FLAG cDNA at 1 and 1/200 dilution, and a negative mock. Rinse cells with serum free DME. Add the DNA solution and incubate 5 hr at 37° C. Remove the medium and add 0.5 ml 10% DMSO in DME for 2.5 min. Remove and wash once with DME. Add 1.5 ml growth medium and incubate overnight.

On day 2, change the medium. On days 3 or 4, the cells are fixed and stained. Rinse the cells twice with Hank's Buffered Saline Solution (HBSS) and fix in 4% paraformaldehyde (PFA)/glucose for 5 min. Wash 3× with HBSS. The slides may be stored at −80° C. after all liquid is removed. For each chamber, 0.5 ml incubations are performed as follows. Add HBSS/saponin(0.1%) with 32 μl/ml of 1M NaN₃ for 20 min. Cells are then washed with HBSS/saponin 1×. Add antibody complex to cells and incubate for 30 min. Wash cells twice with HBSS/saponin. Add second antibody, e.g., Vector anti-mouse antibody, at 1/200 dilution, and incubate for 30 min. Prepare ELISA solution, e.g., Vector Elite ABC horseradish peroxidase solution, and preincubate for 30 min. Use, e.g., 1 drop of solution A (avidin) and 1 drop solution B (biotin) per 2.5 ml HBSS/saponin. Wash cells twice with HBSS/saponin. Add ABC HRP solution and incubate for 30 min. Wash cells twice with HBSS, second wash for 2 min, which closes cells. Then add Vector diaminobenzoic acid (DAB) for 5 to 10 min. Use 2 drops of buffer plus 4 drops DAB plus 2 drops of H₂O₂ per 5 ml of glass distilled water. Carefully remove chamber and rinse slide in water. Air dry for a few minutes, then add 1 drop of Crystal Mount and a cover slip. Bake for 5 min at 85-90° C.

Alternatively, the binding compositions are used to affinity purify or sort out cells expressing the ligand or receptor. See, e.g., Sambrook, et al. or Ausubel et al.

All references cited herein are incorporated herein by reference to the same extent as if each individual publication or patent application was specifically and individually indicated to be incorporated by reference.

Many modification an variations of this invention can be made without departing from its spirit and scope, as will be apparent to those skilled in the art. The specific embodiments described herein are offered by way of example only, and the invention is to be limited only by the terms of the appended claims, along with the full scope of the equivalents to which such claims are entitled.

What is claimed is:

1. A composition of matter selected from the group consisting of:
a) a substantially pure or recombinant CKDLR20.1 protein or peptide exhibiting at least about 85% sequence identity over a length of at least about 12 amino acids to SEQ ID NO: 2;
b) a natural polypeptide sequence CKDLR20.1 of SEQ ID NO: 2;
c) a fusion protein comprising sequence or SEQ ID NO: 2;
d) a substantially pure or recombinant 69A08 protein or peptide exhibiting at least about 85% sequence identity over a length of at least about 12 amino acids to SEQ ID NO: 4 or 6;
e) a natural sequence 69A08 of SEQ ID NO: 4 or 6;
f) a fusion protein comprising sequence of SEQ ID NO: 4 or 6;
g) a substantially pure or recombinant HSD12 protein or peptide exhibiting at least about 85% sequence identity over a length of at least about 12 amino acids to SEQ ID NO: 8;
h) a natural sequence HSD12 of SEQ ID NO: 8; or
i) a fusion protein comprising sequence of SEQ ID NO: 8.

2. A substantially pure or isolated protein comprising:
A) a segment exhibiting sequence identity to a corresponding portion of a CKDLR20.1 of claim 1, wherein:
a) said homology is at least about 90% identity and said portion is at least about 9 amino acids;
b) said homology is at least about 80% identity and said portion is at least about 17 amino acids; or
c) said homology is at least about 70% identity and said portion is at least about 25 amino acids;
B) a segment exhibiting sequence identity to a corresponding portion of a 69A08 receptor of claim 1, wherein:
a) said homology is at least about 90% identity and said portion is at least about 9 amino acids;
b) said homology is at least about 80% identity and said portion is at least about 17 amino acids; or
c) said homology is at least about 70% identity and said portion is at least about 25 amino acids; or
C) a segment exhibiting sequence identity to a corresponding portion of an HSD12 receptor of claim 1, wherein:
a) said homology is at least about 90% identity and said portion is at least about 9 amino acids;
b) said homology is at least about 80% identity and said portion is at least about 17 amino acids; or
c) said homology is at least about 70% identity and said portion is at least about 25 amino acids.

3. The composition of matter of claim 1, wherein said:
A) protein is CKDLR20.1; and
a) said CKDLR20.1 comprises a mature sequence of Table 1; or
b) said protein or peptide:
i) is from a warm blooded animal selected from a mammal, including a rodent;
ii) comprises at least one polypeptide segment of SEQ ID NO: 2;
iii) exhibits a plurality of portions exhibiting said identity;
iv) is a natural allelic variant of CKDLR20.1;
v) has a length at least about 30 amino acids;
vi) exhibits at least two non-overlapping epitopes which are specific for a mammalian CKDLR20.1;
vii) exhibits a sequence identity at least about 90% over a length of at least about 20 amino acids to a rodent CKDLR20.1;
viii) exhibits at least two non-overlapping epitopes which are specific for a rodent CKDLR20.1;
ix) exhibits a sequence identity at least about 90% over a length of at least about 20 amino acids to a rodent CKDLR20.1;
x) is glycosylated;
xi) is a synthetic polypeptide;
xii) is attached to a solid substrate;
xiii) is conjugated to another chemical moiety;
xiv) is a 5-fold or less substitution from natural sequence; or
xv) is a deletion or insertion variant from a natural sequence;
B) protein is 69A08; and
a) said 69A08 comprises a mature sequence of Table 2; or
b) said protein or peptide:
i) is from a warm blooded animal selected from a mammal, including a rodent;
ii) comprises at least one polypeptide segment of SEQ ID NO: 4 or 6;
iii) exhibits a plurality of portions exhibiting said identity;
iv) is a natural allelic variant of rodent 69A08;
v) has a length at least about 30 amino acids;
vi) exhibits at least two non-overlapping epitopes which are specific for a mammalian 69A08;
vii) exhibits a sequence identity at least about 90% over a length of at least about 20 amino acids to a rodent 69A08;
viii) exhibits at least two non-overlapping epitopes which are specific for a rodent 69A08;
ix) exhibits a sequence identity at least about 90% over a length of at least about 20 amino acids to a rodent 69A08;
x) is glycosylated;
xi) is a synthetic polypeptide;
xii) is attached to a solid substrate;
xiii) is conjugated to another chemical moiety;
xiv) is a 5-fold or less substitution from natural sequence; or
xv) is a deletion or insertion variant from a natural sequence; or
C) protein is HSD12; and
a) said HSD12 comprises a mature sequence of Table 3; or
b) said protein or peptide:
i) is from a warm blooded animal selected from a mammal, including a primate;
ii) comprises at least one polypeptide segment of SEQ ID NO: 8;
iii) exhibits a plurality of portions exhibiting said identity;
iv) is a natural allelic variant of HSD12;
v) has a length at least about 30 amino acids;
vi) exhibits at least two non-overlapping epitopes which are specific for a mammalian HSD12;
vii) exhibits a sequence identity at least about 90% over a length of at least about 20 amino acids to a primate HSD12;
viii) exhibits at least two non-overlapping epitopes which are specific for a primate HSD12;
ix) exhibits a sequence identity at least about 90% over a length of at least about 20 amino acids to a primate HSD12;
x) is glycosylated;
xi) is a synthetic polypeptide;
xii) is attached to a solid substrate;
xiii) is conjugated to another chemical moiety;
xiv) is a 5-fold or less substitution from natural sequence; or
xv) is a deletion or insertion variant from a natural sequence;

4. A composition comprising:
a) a sterile CKDLR20.1 protein or peptide of claim 1;
b) said CKDLR20.1 protein or peptide of claim 1 and a carrier, wherein said carrier is:
i) an aqueous compound, including water, saline, and/or buffer; and/or
ii) formulated for oral, rectal, nasal, topical, or parenteral administration;
c) a sterile 69A08 protein or peptide of claim 1;
d) said 69A08 protein or peptide of claim 1 and a carrier, wherein said carrier is:
i) an aqueous compound, including water, saline, and/or buffer; and/or
ii) formulated for oral, rectal, nasal, topical, or parenteral administration;
e) a sterile 69A08 protein or peptide of claim 1; or
f) said HSD12 protein or peptide of claim 1 and a carrier, wherein said carrier is:
i) an aqueous compound, including water, saline, and/or buffer; and/or
ii) formulated for oral, rectal, nasal, topical, or parenteral administration.

5. The fusion protein of claim 1, comprising:
a) mature protein sequence of Table 1;
b) mature protein sequence of Table 2;
c) mature protein sequence of Table 3;
d) a detection or purification tag, including a FLAG, His6, or Ig sequence;
e) sequence of another chemokine protein; or
f) sequence of another G protein coupled receptor protein.

6. A kit comprising:
A) a CKDLR20.1 protein or polypeptide of claim 1, and:
a) a compartment comprising said protein or polypeptide; and/or
b) instructions for use or disposal of reagents in said kit;
B) a 69A08 protein or polypeptide of claim 1, and:
a) a compartment comprising said protein or polypeptide; and/or
b) instructions for use or disposal of reagents in said kit; or
C) an HSD12 protein or polypeptide of claim 1, and:
a) a compartment comprising said protein or polypeptide; and/or
b) instructions for use or disposal of reagents in said kit.

7. A binding compound comprising an antigen binding portion from an antibody, which specifically binds to:
A) a natural CKDLR20.1 protein of claim 1, wherein:
a) said protein is a rodent protein;
b) said binding compound is an Fv, Fab, or Fab2 fragment;
c) said binding compound is conjugated to another chemical moiety; or
d) said antibody:
i) is raised against a peptide sequence of a mature polypeptide comprising sequence of Table 1;
ii) is raised against a mature CKDLR20.1;
iii) is raised to a purified CKDLR20.1;
iv) is immunoselected;
v) is a polyclonal antibody;
vi) binds to a denatured CKDLR20.1;
vii) exhibits a Kd to antigen of at least 30 μM;
viii) is attached to a solid substrate, including a bead or plastic membrane;
ix) is in a sterile composition; or
x) is detectably labeled, including a radioactive or fluorescent label;
B) a natural 69A08 protein of claim 1, wherein:
a) said protein is a rodent protein;
b) said binding compound is an Fv, Fab, or Fab2 fragment;
c) said binding compound is conjugated to another chemical moiety; or
d) said antibody:
i) is raised against a peptide sequence of a mature polypeptide comprising sequence of Table 2;
ii) is raised against a mature 69A08;
iii) is raised to a purified 69A08;
iv) is immunoselected;
v) is a polyclonal antibody;
vi) binds to a denatured 69A08;
vii) exhibits a Kd to antigen of at least 30 μM;
viii) is attached to a solid substrate, including a bead or plastic membrane;
ix) is in a sterile composition; or
x) is detectably labeled, including a radioactive or fluorescent label; or
C) a natural HSD12 protein of claim 1, wherein:
a) said protein is a primate protein;
b) said binding compound is an Fv, Fab, or Fab2 fragment;
c) said binding compound is conjugated to another chemical moiety; or
d) said antibody:
i) is raised against a peptide sequence of a mature polypeptide comprising sequence of Table 3;
ii) is raised against a mature HSD12;
iii) is raised to a purified HSD12;
iv) is immunoselected;
v) is a polyclonal antibody;
vi) binds to a denatured HSD12;
vii) exhibits a Kd to antigen of at least 30 μM;
viii) is attached to a solid substrate, including a bead or plastic membrane;
ix) is in a sterile composition; or
x) is detectably labeled, including a radioactive or fluorescent label; or

8. A kit comprising said binding compound of:
A) claim 7A, and:
a) a compartment comprising said binding compound; and/or
b) instructions for use or disposal of reagents in said kit;
B) claim 7B, and:
a) a compartment comprising said binding compound; and/or
b) instructions for use or disposal of reagents in said kit; or
C) claim 7C, and:
a) a compartment comprising said binding compound; and/or
b) instructions for use or disposal of reagents in said kit.

9. A method of producing an antigen:antibody complex, comprising contacting:
a) a CKDLR20.1 protein or peptide with an antibody of claim 7A;
b) a 69A08 protein or peptide with an antibody of claim 7B; or
c) an HSD12 protein or peptide-with an antibody of claim 7C;
thereby allowing said complex to form.

10. The method of claim 9, wherein:
a) said complex is purified from other chemokine or chemokine receptor;
b) said complex is purified from other antibody;
c) said contacting is with a sample comprising said CKDLR20.1 chemokine antigen;
d) said contacting is with a sample comprising said 69A08 or HSD12 receptor antigen;
e) said contacting allows quantitative detection of said antigen;
f) said contacting is with a sample comprising said antibody; or
g) said contacting allows quantitative detection of said antibody.

11. A composition comprising:
a) a sterile binding compound of claim 7A, or
b) said binding compound of claim 7A and a carrier, wherein said carrier is:
i) an aqueous compound, including water, saline, and/or buffer; and/or
ii) formulated for oral, rectal, nasal, topical, or parenteral administration;
c) a sterile binding compound of claim 7B, or
d) said binding compound of claim 7B and a carrier, wherein said carrier is:
i) an aqueous compound, including water, saline, and/or buffer; and/or
ii) formulated for oral, rectal, nasal, topical, or parenteral administration;
e) a sterile binding compound of claim 7C, or
f) said binding compound of claim 7C and a carrier, wherein said carrier is:
i) an aqueous compound, including water, saline, and/or buffer; and/or
ii) formulated for oral, rectal, nasal, topical, or parenteral administration.

12. An isolated or recombinant nucleic acid encoding a protein or peptide or fusion protein of claim 1, wherein:
A) said protein or polypeptide is CKDLR20.1 and:
a) said CKDLR20.1 protein is from a mammal, including a rodent; or
b) said nucleic acid:
i) encodes an antigenic peptide sequence of Table 1;
ii) encodes a plurality of antigenic peptide sequences of Table 1;
iii) exhibits at least about 80% identity to a natural cDNA encoding said segment;
iv) comprises a stretch exhibiting at least 80% identity to the mature open reading frame from SEQ ID NO: 1;
v) is an expression vector;
vi) further comprises an origin of replication;
vii) is from a natural source;
viii) comprises a detectable label;
ix) comprises synthetic nucleotide sequence;
x) is less than 6 kb, preferably less than 3 kb;
xi) is from a mammal, including a primate;
xii) comprises a natural full length coding sequence;
xiii) is a hybridization probe for a gene encoding said CKDLR20.1 protein; or
xiv) is a PCR primer, PCR product, or mutagenesis primer;
B) said protein or polypeptide is 69A08 and:
a) said 69A08 protein is from a mammal, including a rodent; or
b) said nucleic acid:
i) encodes an antigenic peptide sequence of Table 2;
ii) encodes a plurality of antigenic peptide sequences of Table 2;
iii) exhibits at least about 80% identity to a natural cDNA encoding said segment;
iv) comprises a stretch exhibiting at least 80% identity to the mature open reading frame from SEQ ID NO: 3 or 5;
v) is an expression vector;
vi) further comprises an origin of replication;
vii) is from a natural source;
viii) comprises a detectable label;
ix) comprises synthetic nucleotide sequence;
x) is less than 6 kb, preferably less than 3 kb;
xi) is from a mammal, including a primate;
xii) comprises a natural full length coding sequence;
xiii) is a hybridization probe for a gene encoding said 69A08 protein; or
xiv) is a PCR primer, PCR product, or mutagenesis primer; or
C) said protein or polypeptide is HSD12 and:
a) said HSD12 protein is from a mammal, including a primate; or
b) said nucleic acid:
i) encodes an antigenic peptide sequence of Table 3;
ii) encodes a plurality of antigenic peptide sequences of Table 3;
iii) exhibits at least about 80% identity to a natural cDNA encoding said segment;
iv) comprises a stretch exhibiting at least 80% identity to the mature open reading frame from SEQ ID NO: 7;
v) is an expression vector;
vi) further comprises an origin of replication;
vii) is from a natural source;
viii) comprises a detectable label;
ix) comprises synthetic nucleotide sequence;
x) is less than 6 kb, preferably less than 3 kb;
xi) is from a mammal, including a primate;
xii) comprises a natural full length coding sequence;
xiii) is a hybridization probe for a gene encoding said HSD12 protein; or
xiv) is a PCR primer, PCR product, or mutagenesis primer.

13. A cell or tissue comprising a recombinant nucleic acid of claim 12A, 12B, or 12C.

14. The cell of claim 13, wherein said cell is:
a) a prokaryotic cell;
b) a eukaryotic cell;
c) a bacterial cell;
d) a yeast cell;
e) an insect cell;
f) a mammalian cell;
g) a mouse cell;
h) a primate cell; or
i) a human cell.

15. A kit comprising said nucleic acid:
A) of claim 12A, and:
a) a compartment comprising said nucleic acid;
b) a compartment further comprising a CKDLR20.1 protein or polypeptide; and/or
b) instructions for use or disposal of reagents in said kit;
B) of claim 12B, and:
a) a compartment comprising said nucleic acid;
b) a compartment further comprising a 69A08 protein or polypeptide; and/or
b) instructions for use or disposal of reagents in said kit; or
C) of claim 12C, and:
a) a compartment comprising said nucleic acid;
b) a compartment further comprising an HSD12 protein or polypeptide; and/or
b) instructions for use or disposal of reagents in said kit.

16. A nucleic acid of which:
a) hybridizes under wash conditions of 30° C. and less than 2M salt to SEQ ID NO: 1;
b) exhibits at least about 85% identity over a stretch of at least about 30 nucleotides to a rodent CKDLR20.1;
c) hybridizes under wash conditions of 30° C. and less than 2M salt to SEQ ID NO: 3 or 5;
d) exhibits at least about 85% identity over a stretch of at least about 30 nucleotides to a rodent 69A08;
e) hybridizes under wash conditions of 30° C. and less than 2M salt to SEQ ID NO: 7; or
f) exhibits at least about 85% identity over a stretch of at least about 30 nucleotides to a primate HSD12.

17. The nucleic acid of claim 16, wherein:
a) said wash conditions are at 45° C. and/or 500 mM salt; or
b) said identity is at least 90% and/or said stretch is at least 55 nucleotides.

18. The nucleic acid of claim 17, wherein:
a) said wash conditions are at 55° C. and/or 150 mM salt; or
b) said identity is at least 95% and/or said stretch is at least 75 nucleotides.

19. A method of producing a ligand:receptor complex, comprising contacting:
a) a protein made by expression of a nucleic acid of claim 12A with a G protein coupled receptor;
b) a protein or peptide made by expression of a nucleic acid of claim 12B with a chemokine or ligand; or
c) a protein or peptide made by expression of a nucleic acid of claim 12C with a chemokine or ligand;
thereby allowing said complex to form.

20. The method of claim 19, wherein:
a) said complex results in a Ca++ flux;
b) said G protein coupled receptor is on a cell;
c) said complex results in a physiological change in a cell expressing said receptor or protein;
d) said 69A08 or HSD12 protein is on a cell;
e) said contacting is with a sample comprising a chemical antagonist to block production of said complex; or
f) said contacting allows quantitative detection of said ligand.
